(19) United States
(12) Patent Application Publication
(10) Pub. No.: US 2023/0309861 A1
(43) Pub. Date: Oct. 5, 2023

(54) MOTION SENSOR-MODULATED CARDIAC EPISODE DETECTION AND/OR ALERTING

(71) Applicant: Medtronic, Inc., Minneapolis, MN (US)

(72) Inventors: Michelle M. Galarneau, Fort Collins, CO (US); Wade M. Demmer, Coon Rapids, MN (US); Troy E. Jackson, Rogers, MN (US)

(21) Appl. No.: 18/295,120

(22) Filed: Apr. 3, 2023

Related U.S. Application Data
(60) Provisional application No. 63/362,451, filed on Apr. 4, 2022.

(51) Int. Cl. A61B 5/11; A61N 1/39; A61N 1/362; A61N 1/365; A61B 5/358

(52) U.S. Cl. CPC A61B 5/1117; A61N 1/39622; A61N 1/3624; A61N 1/36585; A61B 5/358

(57) ABSTRACT

An example medical device system includes memory configured to store information relating to an occurrence of a cardiac event in a patient and processing circuitry configured to determine the occurrence of the cardiac event in the patient. The processing circuitry is configured to determine at least one of a fall of the patient, a slumping posture of the patient, or a change from an upright posture to a non-upright posture of the patient and that the cardiac event is associated therewith in time. The processing circuitry is configured to, in response to determining that the cardiac event and the at least one of the fall of the patient, the slumping posture of the patient, or the change from the upright posture to the non-upright posture of the patient are associated in time, modulate a response to the cardiac event.

This application claims the benefit of U.S. Provisional Patent Application 63/362,451, filed Apr. 4, 2022, the entire content of which is incorporated herein by reference.

### TECHNICAL FIELD

The disclosure relates to medical devices, and, more particularly, to medical device systems configured to detect cardiac arrhythmias and/or to treat cardiac arrhythmias with anti-tachyarrhythmia therapy.

### BACKGROUND

Implantable cardioverter defibrillators (ICDs) and implantable artificial pacemakers may provide cardiac pacing therapy to a patient's heart when the natural pacemaker and/or conduction system of the heart fails to provide synchronized atrial and ventricular contractions at rates and intervals sufficient to sustain healthy patient function. Such anti-bradycardial pacing may provide relief from symptoms, or even life support, for a patient. Cardiac pacing may also provide electrical overdrive stimulation, e.g., anti-tachyarrhythmia pacing (ATP) therapy, to suppress or convert tachyarrhythmias, again supplying relief from symptoms and preventing or terminating arrhythmias that could lead to sudden cardiac death. Additionally, ICDs may deliver anti-tachyarrhythmia shocks in response detecting ventricular tachyarrhythmias, e.g., ventricular tachycardia (VT) or ventricular fibrillation (VF), that is not (or likely will not be) terminated by ATP.

### SUMMARY

Implantable medical devices (IMDs), such as ICDs, cardiac resynchronization therapy (CRT) systems, implantable pulse generators (IPGs), temporary ambulatory pacers (TAPs), and external devices, such as external computing devices, may monitor patient parameters and/or deliver therapy, such as ATP therapy or a shock to the heart of a patient. Such therapy may be delivered to advance the heart to refractory thereby terminating a tachyarrhythmia or to treat myocardial infarction.

Such implantable and/or external devices may include an accelerometer. Accelerometer signal(s) may be used to determine a falling event or a posture of a patient. A cardiac event, such as tachyarrhythmia or myocardial infarction, may cause a person to fall or to undergo a significant change in posture, such as from an upright posture to a slumping posture or non-upright posture. As a falling event or significant posture change in a cardiac patient may accompany a cardiac episode (which may also be referred to herein as a cardiac event), it may be beneficial to monitor falling events or significant posture changes in such patients and modulate or change behavior of an IMD or external device, such as delivery of therapy and/or prioritization and/or content of alerts, based on such falling or significant posture change events.

Tachyarrhythmia patients may be more prone to falling during tachyarrhythmia events, such as a tachycardia. Similarly, patients may be more prone to falling during a myocardial infarction event. An IMD or an external device may utilize the accelerometer signal(s) to enhance detection of cardiac arrhythmias or episodes to improve the subsequent therapy the IMD may deliver and/or to provide more accurate/detailed alerting or timely transmission of relevant episode information to stakeholders, such as the patient, a caregiver, a clinician, emergency medical technicians, or the like.

The systems and techniques of this disclosure provide for posture-modulated activities of an IMD such as posture-modulated cardiac event detection and/or treatment, and posture-modulated alert urgency and/or content determinations. While this disclosure is primarily focused on using accelerometer signal(s) to determine changes in posture or a fall of a patient, the use of other accelerometer-derived metrics in addition to or in lieu of posture or a fall, such as activity and “spikes” in motion, are included in the scope of this disclosure. The systems and techniques described herein may be used to deliver tachycardia or other therapy in a more timely manner, may add value to the remote monitoring capabilities of IMDs, may enhance the value of certain IMDs, and may improve the myocardial infarction patient care pathway. By collecting more contextual data around a detected cardiac episode, e.g., whether the patient fell and/or had a significant posture change associated in time with the detected cardiac episode, the IMD may improve the sensitivity and/or specificity of any therapy being delivered, which may provide for better patient outcomes. In addition, falls, postural changes, or other behaviors common with myocardial infarction, might allow for enhanced therapy, diagnosis, and alerting for these patients. As such this disclosure includes technical solutions to technical problems and provides benefits over traditional IMDs.

In one example, this disclosure is directed to a method including determining, by a medical device system, an occurrence of a cardiac event in a patient; determining, by the medical device system and based at least in part on at least one accelerometer signal, at least one of a fall of the patient, a slumping posture of the patient, or a change from an upright posture to a non-upright posture of the patient; determining, by the medical device system, that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulating, by the medical device system, a response to the cardiac event.

In another example, this disclosure is directed to medical device system including memory configured to store information relating to an occurrence of a cardiac event in a patient; and processing circuitry configured to: determine the occurrence of the cardiac event in the patient; determine, based at least in part on at least one accelerometer signal, at least one of a fall of the patient, a slumping posture of the patient, or a change from an upright posture to a non-upright posture of the patient; determine that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulate a response to the cardiac event.

In a further example, this disclosure is directed to a non-transitory computer-readable storage medium having stored thereon instructions that, when executed, cause processing circuitry to: determine the occurrence of the cardiac event in the patient; determine, based at least in part on at least one accelerometer signal, at least one of a fall of the patient, a slumping posture of the patient, or a change from an upright posture to a non-upright posture of the patient; determine that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulate, by the medical device system, a response to the cardiac event.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1A is a front view of a patient implanted with an example implantable medical device system.

FIG. 1B is a side view of the patient implanted with the implantable medical device system of FIG. 1A.

FIG. 1C is a transverse view the patient implanted with the implantable medical device system of FIGS. 1A and 1B.

FIG. 2 is a conceptual drawing illustrating an example configuration of the intracardiovascular pacing device (IPD) of the implantable medical device system of FIGS. 1A-1C.

FIG. 3 is a schematic diagram of another example implantable medical device system in conjunction with a patient.

FIG. 4 is a conceptual drawing illustrating an example configuration of the insertable cardiac monitor (ICM) of the implantable medical device system of FIG. 3.

FIG. 5 is a functional block diagram illustrating an example configuration of the IPD of FIGS. 1A-1C and 2.

FIG. 6 is a functional block diagram illustrating an example configuration of the ICD of the implantable medical device system of FIGS. 1A-1C.

FIG. 7 is a functional block diagram illustrating an example configuration of the external device of FIGS. 1A-1C and FIG. 3.

FIG. 8 is a functional block diagram illustrating an example configuration of the pacemaker/cardioverter/defibrillator (PCD) of the implantable medical device system of FIG. 3.

FIG. 9 is a functional block diagram illustrating an example configuration of the ICM of FIGS. 3-4.

FIG. 10 is a flowchart illustrating example posture or fall based techniques for modulating a response to a detected cardiac event according to this disclosure.

### DETAILED DESCRIPTION

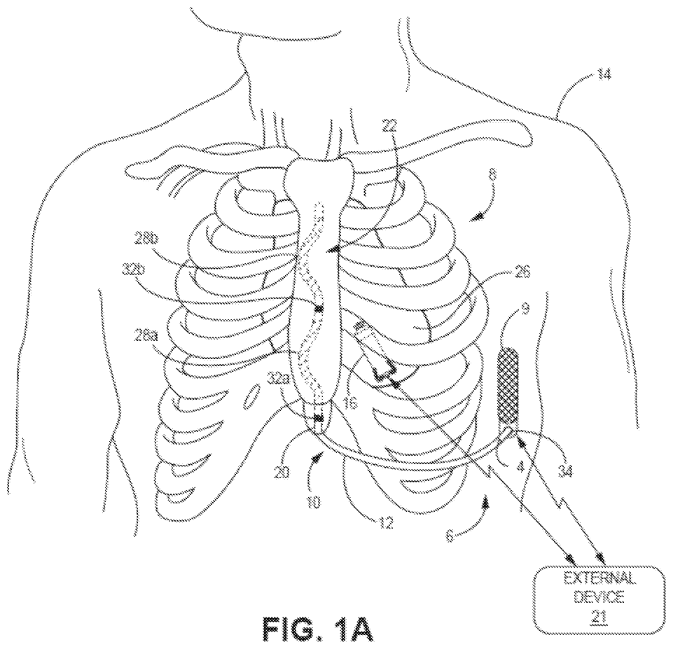

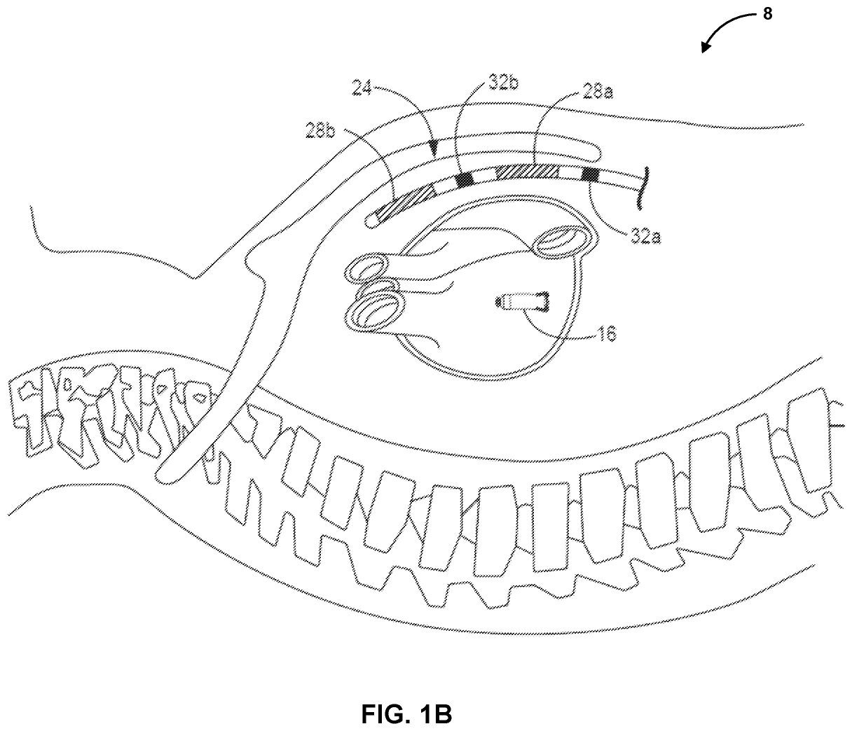

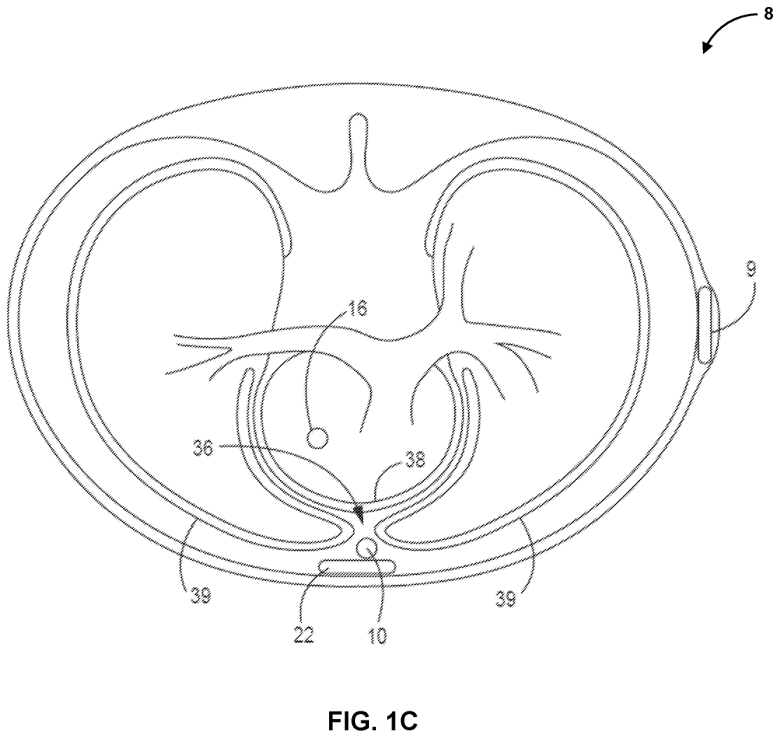

FIGS. 1A-1C are conceptual diagrams illustrating various views of an example implantable medical device system 8. The system 8 includes an extracardiovascular ICD system 6, including ICD 9 connected to a medical electrical lead 10, and IPD 16 configured in accordance with the principles of the present application. FIG. 1A is a front view of a patient 14 implanted with the medical device system 8. FIG. 1B is a side view of patient 14 implanted with the medical device system 8. FIG. 1C is a transverse view of patient 14 implanted with the medical device system 8.

The ICD 9 may include a housing that forms a hermetic seal that protects components of the ICD 9. The housing of the ICD 9 may be formed of a conductive material, such as titanium or titanium alloy, which may function as a housing electrode (sometimes referred to as a can electrode). In other examples, the ICD 9 may be formed to have or may include one or more electrodes on the outermost portion of the housing. The ICD 9 may also include a connector assembly (also referred to as a connector block or header) that includes electrical feedthroughs through which electrical connections are made between conductors of lead 10 and electronic components included within the housing of the ICD 9. As will be described in further detail herein, housing may house one or more processors, memories, transmitters, receivers, sensors, sensing circuitry, therapy delivery circuitry, power sources and other appropriate components. The housing is configured to be implanted in a patient, such as patient 14.

ICD 9 is implanted extra-thoracically on the left side of patient 14, e.g., under the skin and outside the ribcage (subcutaneously or submuscularly). ICD 9 may, in some instances, be implanted between the left posterior axillary line and the left anterior axillary line of patient 14. ICD 9 may, however, be implanted at other extra-thoracic locations on patient 14 as described later.

Lead 10 may include an elongated lead body 12 sized to be implanted in an extracardiovascular location proximate the heart, e.g., intra-thoracically (as illustrated in FIGS. 1A-C), subcutaneously, or intercostally for example. In the illustrated example, lead body 12 extends superiorly intra-thoracically underneath the sternum, in a direction substantially parallel to the sternum. In one example, the distal portion 24 of lead 10 may reside in a substernal location such distal portion 24 of lead 10 extends superior along the posterior side of the sternum substantially within the anterior mediastinum 36. Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the internal thoracic vein. Lead 10 may be implanted at other locations, such as over the sternum, offset to the right of the sternum, angled lateral from the proximal or distal end of the sternum, or the like. In other examples, distal portion of lead 10 may reside within a blood vessel within the substernal location, such as within an internal thoracic artery, an internal thoracic vein, an intercostal vein, the superior epigastric vein, or the azygos, hemiazygos, or accessory hemiazygos veins, as examples. In this manner, lead 10 remains outside the heart in an extra-cardiac location.

Lead body 12 may have a generally tubular or cylindrical shape and may define a diameter of approximately 3-9 French (Fr), however, lead bodies of less than 3 Fr and more than 9 Fr may also be utilized. In another configuration, lead body 12 may have a flat, ribbon, or paddle shape with solid, woven filament, or metal mesh structure, along at least a portion of the length of lead body 12. In such an example, the width across lead body 12 may be between 1-3.5 mm. Other lead body designs may be used without departing from the scope of this application.

Lead body 12 of lead 10 may be formed from a non-conductive material, including silicone, polyurethane, fluoropolymers, mixtures thereof, and other appropriate materials, and shaped to form one or more lumens (not shown), however, the techniques are not limited to such constructions. Distal portion 24 may be fabricated to be biased in a desired configuration, or alternatively, may be manipulated by the user into the desired configuration. For example, distal portion 24 may be composed of a malleable material such that the user can manipulate distal portion 24 into a desired configuration where it remains until manipulated to a different configuration. However, distal portion 24 may take on different configurations, including a straight configuration, circular configuration or any of a number of configurations.

Lead body 12 may include a proximal end 4 and a distal portion 24 configured to deliver electrical energy to the heart or sense electrical energy of the heart. Distal portion 24 may be anchored to a desired positioned within the patient, for example, substernally or subcutaneously by, for example, suturing distal portion 24 to the patient's musculature, tissue, or bone at the xiphoid process entry site. Alternatively, distal portion 24 may be anchored to the patient or through the use of rigid tines, prongs, barbs, clips, screws, and/or other projecting elements or flanges, disks, pliant tines, flaps, porous structures such as a mesh-like element and metallic or non-metallic scaffolds that facilitate tissue growth for engagement, bio-adhesive surfaces, and/or any other non-piercing elements.

Distal portion 24 includes defibrillation electrode 28 configured to deliver a cardioversion/defibrillation shock to the patient's heart. Defibrillation electrode 28 may include a plurality of sections or segments 28a and 28b spaced a distance apart from each other along the length of distal portion 24. The defibrillation electrode segments 28a and 28b may be a disposed around or within lead body 12 of distal portion 24, or alternatively, may be embedded within the wall of lead body 12. In one configuration, defibrillation electrode segments 28a and 28b may each be a coil electrode formed by a conductor. The conductor may be formed of one or more conductive polymers, ceramics, metal-polymer composites, semiconductors, metals or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of the defibrillation electrodes segments 28a and 28b may be a flat ribbon electrode, a paddle electrode, a braided or woven electrode, a mesh electrode, a directional electrode, a patch electrode or another type of electrode configured to deliver a cardioversion/defibrillation shock to the patient's heart.

In one configuration, the defibrillation electrode segments 28a and 28b are spaced approximately 0.25-4.5 cm, and in some instances between 1-3 cm apart from each other. In another configuration, the defibrillation electrode segments 28a and 28b are spaced approximately 0.25-1.5 cm apart from each other. In a further configuration, the defibrillation electrode segments 28a and 28b are spaced approximately 1.5-4.5 cm apart from each other. In the configuration shown in FIGS. 1A-1C, the defibrillation electrode segments 28a and 28b span a substantial part of distal portion 24. Each of the defibrillation electrode segments 28a and 28b may be between approximately 1-10 cm in length and, more preferably, between 2-6 cm in length and, even more preferably, between 3-5 cm in length. However, lengths of greater than 10 cm and less than 1 cm may be utilized without departing from the scope of this disclosure. A total length of defibrillation electrode 28 (e.g., length of the two segments 28a and 28b combined) may vary depending on a number of variables. The defibrillation electrode 28 may, in one example, have a total length of between approximately 5-10 cm. However, the defibrillation electrode segments 28a and 28b may have a total length less than 5 cm and greater than 10 cm in other examples. In some instances, defibrillation segments 28a and 28b may be approximately the same length or, alternatively, different lengths.

The defibrillation electrode segments 28a and 28b may be electrically connected to one or more conductors, which may be disposed in the body wall of lead body 12 or may alternatively be disposed in one or more insulated lumens (not shown) defined by lead body 12. In an example configuration, each of the defibrillation electrode segments 28a and 28b is connected to a common conductor such that a voltage may be applied simultaneously to all the defibrillation electrode segments 28a and 28b to deliver a defibrillation shock to a patient's heart. In other configurations, the defibrillation electrode segments 28a and 28b may be attached to separate conductors such that each defibrillation electrode segment 28 a or 28b may apply a voltage independent of the other defibrillation electrode segments 28a or 28b. In this case, ICD 9 or lead 10 may include one or more switches or other mechanisms to electrically connect the defibrillation electrode segments together to function as a common polarity electrode such that a voltage may be applied simultaneously to all the defibrillation electrode segments 28a and 28b in addition to being able to independently apply a voltage.

In one example, the distance between the closest defibrillation electrode segment 28a and 28b and electrodes 32a and 32b is greater than or equal to 2 mm and less than or equal to 1.5 cm. In another example, electrodes 32a and 32b may be spaced apart from the closest one of defibrillation electrode segments 28a and 28b by greater than or equal to 5 mm and less than or equal to 1 cm. In a further example, electrodes 32a and 32b may be spaced apart from the closest one of defibrillation electrode segments 28a and 28b by greater than or equal to 6 mm and less than or equal to 8 mm.

Electrodes 32a and 32b may be configured to deliver low-voltage electrical pulses to the heart and/or may sense a cardiac electrical activity, e.g., depolarization and repolarization of the heart. As such, electrodes 32a and 32b may be referred to herein as pace/sense electrodes 32a and 32b. In one configuration, electrodes 32a and 32b are ring electrodes. However, in other configurations the electrodes 32a and 32b may be any of a number of different types of electrodes, including ring electrodes, short coil electrodes, paddle electrodes, hemispherical electrodes, directional electrodes, or the like. Electrodes 32a and 32b may be the same or different types of electrodes. Electrodes 32a and 32b may be electrically isolated from an adjacent defibrillation segment 28a or 28b by including an electrically insulating layer of material between electrodes 32a and 32b and the adjacent defibrillation segments 28a and 28b. Each electrode 32a or 32b may have its own separate conductor such that a voltage may be applied to each electrode independently from another electrode 32a or 32b in distal portion 24. In other configurations, each electrode 32a or 32b may be coupled to a common conductor such that each electrode 32a or 32b may apply a voltage simultaneously.

Proximal end 4 of lead body 12 may include one or more connectors 34 to electrically couple lead 10 to ICD 9 subcutaneously implanted within the patient, for example, under the left armpit of the patient. The ICD 9 may include a housing that forms a hermetic seal which protects the components of ICD 9. The housing of ICD 9 may be formed of a conductive material, such as titanium or titanium alloy, which may function as a housing electrode for a particular therapy vector between the housing and distal portion 24. ICD 9 may also include a connector assembly that includes electrical feedthroughs through which electrical connections are made between the one or more connectors 34 of lead 10 and the electronic components included within the housing. The housing of ICD 9 may house one or more processors, memories, transmitters, receivers, sensors, sensing circuitry, therapy circuitry, power sources (capacitors and batteries) and/or other appropriate components. The components of ICD 9 may generate and deliver electrical stimulation therapy such as ATP.

The inclusion of electrodes 32a and 32b adjacent to defibrillation electrode segments 28a and 28b provides a number of therapy vectors for the delivery of electrical stimulation therapy to the heart. For example, as shown in FIGS. 1A-1C, at least a portion of the defibrillation electrode 28 and one of the electrodes 32a and 32b may be disposed over the right ventricle, or any chamber of the heart, such that pacing pulses and defibrillation shocks may be delivered to the heart. The housing of ICD 9 may be charged with or function as a polarity different than the polarity of the one or more defibrillation electrode segments 28a and 28b and/or electrodes 32a and 32b such that electrical energy may be delivered between the housing and the defibrillation electrode segment(s) 28a and 28b and/or electrode(s) 32a and 32b to the heart. Each defibrillation electrode segment 28a or 28b may have the same polarity as every other defibrillation electrode segment 28a or 28b when a voltage is applied to it such that a defibrillation shock may be delivered from the entirety of the defibrillation electrode 28. In examples in which defibrillation electrode segments 28a and 28b are electrically connected to a common conductor within lead body 12, this is the only configuration of defibrillation electrode segments 28a and 28b. However, in other examples, defibrillation electrode segments 28a and 28b may be coupled to separate conductors within lead body 12 and may therefore each have different polarities such that electrical energy may flow between defibrillation electrode segments 28a and 28b (or between one of defibrillation electrode segments 28a and 28b and one or more pace/sense electrodes 32a and 32b or the housing electrode) to provide pacing therapy and/or to sense cardiac depolarizations. In this case, the defibrillation electrode segments 28a and 28b may still be electrically coupled together (e.g., via one or more switches within ICD 9) to have the same polarity to deliver a defibrillation shock from the entirety of the defibrillation electrode 28.

Additionally, each electrode 32a and 32b may be configured to conduct electrical pulses directly to the heart, or sense a cardiac depolarization between adjacent defibrillation electrode segments 28a and 28b, whether disposed on the same defibrillation electrode segment 28a or 28b or on other defibrillation electrode segment 28a or 28b, and/or between proximate electrodes 32a and 32b. Additionally, electrodes 32a and 32b may conduct electrical pulses between one another, e.g., between one of electrodes 32a and 32b and an inferior and superior electrode 32a and 32b, between one of electrodes 32a and 32b and the housing electrode, or between a plurality of electrodes 32a and 32b (at the same polarity) and the housing electrode at the opposite polarity. As such, each electrode 32a or 32b may have the same polarity as every other electrode 32a or 32b or alternatively, may have different polarities such that different therapy vectors can be utilized to deliver pacing pulses to the heart.

IPD 16 may be implanted within a heart 26 of patient 14. In the example of FIGS. 1A-1C, IPD 16 is implanted within right ventricle of heart 26 to sense electrical activity of heart 26 and deliver pacing therapy, e.g., ATP therapy, to heart 26. IPD 16 may be attached to an interior wall of the right ventricle of heart 26 via one or more fixation elements that penetrate the tissue. These fixation elements may secure IPD 16 to the cardiac tissue and retain an electrode (e.g., a cathode or an anode) in contact with the cardiac tissue. However, in other examples, system 8 may include additional pacing devices 16 within respective chambers of heart 26 (e.g., right or left atrium and/or left ventricle). In further examples, a cardiac pacing device configured similarly to IPD 16 may be attached to an external surface of heart 26 (e.g., in contact with the epicardium) such that the pacing device is disposed outside of heart 26.

IPD 16 may be capable sensing electrical signals using the electrodes carried on the housing of IPD 16. These electrical signals may be electrical signals generated by cardiac muscle and indicative of depolarizations and repolarizations of heart 26 at various times during the cardiac cycle. IPD 16 may analyze the sensed electrical signals to detect tachyarrhythmias, such as ventricular tachycardia or ventricular fibrillation. In response to detecting the tachyarrhythmia, IPD 16 may, e.g., depending on the type of tachyarrhythmia, begin to deliver ATP therapy via the electrodes of IPD 16. In some examples, such therapy, or the initiation thereof, may be modulated based on whether or not IPD 16, ICD 9, and/or external device 21 has detected a fall, a slumping posture, or a change from an upright posture to a non-upright posture in patient 14.

In some examples, IPD 16 and ICD 9 may be configured to communicate with one another, e.g., via radio-frequency communication, conductive or galvanic communication or other type of communication, to cooperate with one another. For example, IPD 16 and ICD 9 may communicate information, such as sense signals, accelerometer signals, and/or delivered signals, and may coordinate to establish pacing and/or sensing vectors between respective electrodes on ICD 9, IPD 16, and/or lead 10. IPD 16 and ICD 9 may be configured for one-way or two-way communication.

In other examples, IPD 16 and ICD 9 are not configured to communicate with each other. In such examples, each of IPD 16 and ICD 9 may independently monitor the electrical activity of heart, accelerometer signals, and deliver therapy in response to detecting arrhythmia and whether the accelerometer signals indicate that patient 14 has fallen, has entered a slumping posture, or has had a change from an upright posture to a non-upright posture. In such examples, one or both of IPD 16 and ICD 9 may be configured to detect activity of, e.g., delivery of therapy by, the other. In this manner, delivery of therapies by IPD 16 and ICD 9 may be coordinated without conventional uni- or bi-directional communication between the devices. In some examples, a medical system according to the techniques of this disclosure may include one device, such as one of ICD 9, IPD 16, or another IMD, and may not include the other device.

According to the techniques of this disclosure, system 8 (e.g., ICD 9, IPD 16, external device 21, or any combination thereof) may determine the occurrence of a cardiac event in patient 14. For example, system 8 may determine the occurrence of tachyarrhythmia, a pause in heartbeat, or myocardial infarction in patient 14. System 8 may determine, based at least in part on at least one accelerometer signal, at least one of a fall of patient 14, a slumping posture of patient 14, or a change from an upright posture to a non-upright posture of patient 14. System 8 may determine that the cardiac event and the at least one of the fall of patient 14, the slumping posture of patient 14, or the change from the upright posture to the non-upright posture of patient 14 are associated in time. For example, system 8 may determine that the fall of patient 14, the slumping posture of patient 14, or the change from the upright posture to the non-upright posture of patient 14 occurred during the cardiac event, within a first predetermined time period before the cardiac event, or within a second predetermined time period after the cardiac event. For example, the first predetermined time period may be in the range of 0 seconds to 24 hours and the second predetermined time period may be in the range of 5 seconds to 5 minutes. In response to determining that the cardiac event and the at least one of the fall of the patient, the slumping posture of a patient, or the change from the upright posture to the non-upright posture of the patient are associated in time, system 8 may modulate a response to the cardiac event. In some examples, the modulated response may be a change in how, when, or under what circumstances therapy is delivered. In some examples, the modulated response may be how an indication of the cardiac event, such as an alert, may be generated and delivered.

For example, a three-axis accelerometer (or three separate single-axis accelerometers) may produce a signal along each of three accelerometer axes: the x axis, the y axis and the z axis. The signals from such an accelerometer may be used to determine a posture of patient 14. For example, such signals may define a vector corresponding with the posture of patient 14. Changes in the direction of the vector within a short time period, such as a few seconds, may indicate the posture of patient 14 has changed. Additionally, detected changes in posture accompanied by a spike in the accelerometer signal(s) due to impact with the ground or acceleration during a fall, may indicate that patient 14 has fallen. More details on how accelerometer signals may be used to determine posture changes of a patient, such as patient 14, may be found in U.S. Publication 2021/0085202-A1,entitled, “AUTOMATIC DETECTION OF BODY PLANES OF ROTATION,” the entire content of which is herein incorporated by reference. More details on how to determine a patient, such as patient 14, has fallen may be found in U.S. Patent Publication 2020/0380840-A1, entitled, “MEDICAL DEVICE FOR FALL DETECTION,” the entire content of which is herein incorporated by reference.

Posture-modulated arrhythmia treatment of ventricular tachycardia episodes is now discussed. System 8 may detect a fall, a slumping posture, or a change from an upright posture to a non-upright posture based on one or more accelerometer signals during an in-progress ventricular tachycardia episode. System 8 may determine a ventricular tachyarrhythmia episode is occurring based on a sensed electrocardiogram signal. For example, during a ventricular tachyarrhythmia episode, the electrocardiogram signal may increase in speed and become erratic. System 8 may determine the patient has fallen, entered a slumping posture, or changed from an upright posture to a non-upright posture based on accelerometer signals. In response to determining that a ventricular tachyarrhythmia episode is occurring and determining the patient has fallen, entered a slumping posture, or changed from an upright posture to a non-upright posture during the ventricular tachyarrhythmia episode (or shortly thereafter), system 8 may deliver a shock.

In some examples, system 8 may include rules, such as part of a cardiac rhythm discrimination algorithm, that must be fulfilled prior to system 8 delivering a shock so as to reduce or eliminate the delivery of a shock when patient 14 may not need a shock. For example, the rules may protect against possible misclassification of a “do not treat” state as a “do treat” state. For example, there are times when patient 14 may have a high heartrate, but other physiological parameters indicate that treatment is not necessary (e.g., the patient is undergoing intense exercise, the patient has a moderate electrolyte imbalance, or the patient is in an environment with a strong radiated electric field). Such rules may be based on the state of one or more physiological parameters of the patient. Such physiological parameters of the patient may be sensed or measured by sensors of system 8. In the case system 8 has such rules and detects a fall or significant posture change, system 8 may override such rules and deliver a shock whether or not sensed physiological parameters meet such rules. In other examples, rather than override the rules, system 8 may relax the rules making it more likely that system 8 will deliver the shock to the patient. Relaxing or overriding the rules may be an example of modulating the response to the cardiac event.

In some examples, rather than immediately delivering the shock, system 8 may first determine that the patient has stopped moving for a predetermined period of time, e.g., based on the accelerometer signal(s), or is in a lying posture after falling, e.g., based on the accelerometer signal(s). In some examples, this predetermined period of time may be greater than 0 seconds and less than or equal to 15 seconds. In some examples, the predetermined period of time may be on the order of 10-15 seconds. In some examples, system 8 may modulate the operation of ATP therapy and begin administering therapy upon determining the patient has stopped moving or is in a lying posture after falling.

In some examples, system 8 may detect a fall, a slumping posture, or a change in posture from an upright posture to a non-upright posture, and relax ventricular tachycardia onset detection criteria, thereby making it easier to detect and treat ventricular tachycardia earlier. To detect a slumping posture, system 8 may detect a relatively slow change in the angle of the upper body of patient 14 from upright over time that is not accompanied by a spike in acceleration of patient 14. For example, system 8 may include criteria or rules that provide a decision threshold as to whether system 8 determines that ventricular tachyarrhythmia is occurring. By relaxing the ventricular tachycardia onset detection criteria, system 8 may bias any such decision threshold towards delivering treatment. Such a feature may be particularly valuable with an IMD that does not include the ability to deliver back-up shock therapy.

In some examples, system 8 may modulate the criteria or rules in such a manner as to deliver therapy, such as a shock or ATP therapy to patient 14, at a different time than system 8 would have delivered such therapy if system 8 did not modulate the criteria or rules. For example, system 8 may modulate the criteria or rules to deliver therapy sooner or later than system 8 otherwise would have delivered the therapy.

In some examples, system 8 may determine that the patient has stopped moving, e.g., based on the accelerometer signal(s) for a predetermined period of time, or is in a lying posture after falling, e.g., based on the accelerometer signal(s), prior to delivering the shock. In some examples, this predetermined period of time may be greater than 0 seconds and less than or equal to 15 seconds. In some examples, the predetermined period of time may be on the order of 10-15 seconds. For example, system 8 may monitor motion of the patient or a posture of the patient after the falling event and only deliver the shock if the patient has not substantially moved or is still in a lying posture after falling for a predetermined period of time. In some examples, this predetermined period of time may be greater than 0 seconds and less than or equal to 15 seconds. In some examples, the predetermined period of time may be on the order of 10-15 seconds. By monitoring the motion or posture of the patient after the falling event and only delivering the shock if the patient has not substantially moved or is still in the lying posture after the predetermined period of time, system 8 may reduce the likelihood an IMD delivers a shock when the patient may not need the shock.

Posture-modulated arrhythmia treatment of atrial tachyarrhythmia, e.g., atrial tachycardia or atrial fibrillation, episodes is now discussed. In some examples, system 8 may detect a fall of a patient during an in-progress atrial tachyarrhythmia. For example, system 8 may determine an atrial tachyarrhythmia episode is occurring based on a sensed electrocardiogram signal. For example, during an atrial tachyarrhythmia episode, the electrocardiogram signal may increase in speed and become erratic. In response to detecting a fall, a slumping posture, or a change from an upright posture to a non-upright posture of patient 14, system 8 may modulate the operation of ATP therapy. For example, an IMD of system 8 may fire ATP therapy sooner than the IMD may otherwise. In some examples, system 8 may detect a fall, a slumping posture, or a change from an upright posture to a non-upright posture of patient 14 and relax existing atrial tachyarrhythmia onset detection criteria which may make it easier to detect atrial tachyarrhythmias sooner than otherwise (e.g., sooner than system 8 may detect atrial tachyarrhythmias under the existing atrial tachyarrhythmia onset detection criteria if the existing atrial tachyarrhythmia onset detection criteria were not relaxed). Such a feature may provide value, for example, if an IMD does not include back-up shock therapy.

Posture-modulated alert urgency is now discussed. Some IMDs which may include IPD 16, ICD 9, or a TAP (not shown for simplicity purposes), which may be part of system 8, may be configured to detect a pause in a heartbeat or cardiac rhythm. For example, a system 8 may monitor an electrocardiogram (ECG) signal, an audio signal corresponding with a heartbeat, or other sensor signal indicative of a pause in a heartbeat. A pause in the heartbeat may be defined as an interruption in a regular heartbeat, such as an interruption in ventricular depolarization which may be represented by prolonged R-R interval in the ECG or EGM signal. When system 8 determines a pause in the heartbeat of the patient based on sensor signal(s), system 8 may determine a first posture before the pause based on accelerometer signal(s) before the pause. In some examples, system 8 may determine the first posture prior to determining the pause in the heartbeat. System 8 may determine a second posture after the pause based on accelerometer signal(s) after the pause. System 8 may store the first posture and the second posture in memory. For example, system 8 may store the first posture as upright and may store the second posture as side lying. Additionally, or alternatively, system 8 may store posture counts or signals over time associated with the first posture and the second posture. For example, the posture counts may indicate a length of time the patient is in the first posture and a length of time the patient is in the second posture. Additionally, or alternatively, system 8 may store the length of time the patient is in the first posture and the length of time the patient is in the second posture. In some examples, system 8 may store whether a fall, a slumping posture, or a change from an upright posture to a non-upright posture of patient 14 was detected during or after the pause in memory. In some examples, system 8 may only store the fall, slumping posture, or change from an upright posture to a non-upright posture of patient 14 occurring after the pause in memory if the fall, slumping posture, or change from an upright posture to a non-upright posture of patient 14 occurs within a predetermined time period of the pause, such as 0 to 30 seconds.

In some situations, a change in posture may result in lower amplitude signals reaching the sensing circuitry, for example, due to a change in the distance from the electrodes, microphone, etc., to the heart or a change in tissue contracted by the electrodes, microphone, etc. This change could result in undersensing caused by lower signal amplitudes not being sensed by the sensing circuitry in the new posture. Such undersensing may appear to be a pause in the heartbeat, while actually not representing a pause in the heartbeat. To avoid, or lessen the chances of, determining a pause in the heartbeat when there is actually no pause in the heartbeat, sensing circuitry of system 8 may include a second sensing channel for sensing, at higher sensitivity level than a first sensing channel (e.g., the second sensing channel has a lower sensing threshold than the first sensing channel). When a change in posture is detected, system 8 may use this second sensing channel to sense the heartbeat (or pause in the heartbeat) or system 8 may use the second sensing channel to verify (or reject) a sensed pause in the heartbeat. In some examples, system 8 may use another sensing source to verify (or reject) a sensed pause in the heartbeat. For example, if system 8 uses an ECG signal to determine a pause in the heartbeat, system 8 may attempt to verify the pause using an audio signal. In some examples, system 8 may use one or more accelerometer signals, filtered for heart sounds and/or cardiac motion, to verify (or reject) the suspected pause in the heartbeat. In some examples, system 8 may include a buffer of previous sensed signals which system 8 may look back to in an attempt to verify (or reject) a suspected pause in heartbeat.

In some examples, system 8 may generate an indication for output, such as an alert for transmission, to a remote monitoring device. System 8 may then transmit the indication to the remote monitoring device. In some examples, external device 21 may be the remote monitoring device. In other examples, another device may be the remote monitoring device, such as a cloud-based monitoring service. The indication may include information relating to all detected pauses. For example, the indication may include a time of the pause, a length of the pause, the first posture information, the second posture information, whether there was a fall involved, etc. In some examples, system 8 may determine whether the first posture was an upright posture and the second posture was a non-upright posture, whether the second posture was a slumping posture, or whether a fall occurred associated with the pause and only transmit the indication for pauses in which one of these conditions is met.

In some examples, system 8 may determine that the second posture is an upright posture. In such an example, system 8 may generate an indication for output that is directed to the patient or a caregiver. For example, system 8 may generate an alert which may be an audio alert or visual alert, such as a flashing light or a written alert (e.g., short message service (SMS) message, or email) which system 8 may transmit to a patient device or caregiver device, such as a cellular phone. The cellular phone may play the alert or display the alert. Such an alert may include, for example, “A pause in your heartbeat was detected. Seek medical attention.” In some examples, if the second posture is lying, IMD may generate an indication for output to an emergency medical service.

In some examples, the intended recipient of indications for outputs, such as alerts, may be at least in part based on a living situation of patient 14 (e.g., a type of place in which patient 14 is living). For example, if patient 14 is living at home, the indication may be output to a patient device such as a cellular phone, smart television, smart speaker, or the like (which may be examples of external device 21). If patient 14 is living in a senior community, the indication may be output to a patient device, a neighbor's device, or an employee or contractor of the senior community, or the like (which may be examples of external device 21) If patient 14 is living in an assisted living facility, the indication may be output to the patient device, a caregiver device, or the like (which may be examples of external device 21). If the patient is in a nursing home, the indication may be output to a caregiver device or an emergency medical service (which may be examples of external device 21).

In some examples, a patient, caregiver, clinician, or other user may program one or more of the devices of system 8 via external device 21 to indicate preferences for under what circumstance, when, or to whom an indication, such as an alert, should be delivered.

For example, a user may program system 8 to not deliver alerts during sleeping hours so as not to disturb patient 14 or others, or to deliver all alerts to a caregiver, family member, or friend, in addition to, any other recipient of an alert. External device 21 may store such preferences in memory and, prior to delivering the indication, verify that delivering the indication does not violate the preferences.

Posture-modulated alert urgency/content for tachyarrhythmia (atrial or ventricular) episode detection is now discussed. System 8 may detect a tachyarrhythmia episode. System 8 may determine, and store, posture counts or signals over the course of the detected episode. System 8 may also determine, based on accelerometer signal(s) whether a fall, a slumping posture, or a change from an upright posture to a non-upright posture, of patient 14 occurred during, within a first predetermined time period before, or within a second predetermined time period after the detected episode. The first predetermined time period may be on the order of 0 seconds to 24 hours and the second predetermined time period may be on the order of 5 second to 5 minutes. If a fall, a slumping posture, or a change from an upright posture to a non-upright posture, has occurred, system 8 may determine how far (e.g., in terms of time, such as x seconds) into the episode the fall, the slumping posture, or the change from the upright posture to the non-upright posture occurred, and store this information in memory.

In some examples, whether the fall, the slumping posture, or the change from the upright posture to the non-upright posture occurred before or after the cardiac event, such as tachyarrhythmia, may be indicative of a particular patient health event. For example, if the tachyarrhythmia event occurs before the fall, the slumping posture, or the change from the upright posture to the non-upright posture, the cardiac event, such as the tachyarrhythmia event, may be more likely to have caused the fall. However, if the fall, the slumping posture, or the change from the upright posture to the non-upright posture occurs before the tachyarrhythmia event, then the patient may have had a different health event that precipitated the cardiac event, such as a stroke. As such, system 8 may store an order of events in memory and generate an indication including the order of events. In some examples, the indication may include instructions for medical treatment and/or a predicted diagnosis which may be provided to an emergency medical service, caregiver, clinician, or the like.

A patient's posture or activity after a fall may be an indicator of post-fall acute recovery. As such, system 8 may determine a post-fall posture or post-fall activity after determining the fall occurred based on accelerometer signal(s). System 8 may store an indication of the post-fall posture or post-fall activity after the fall. System 8 may generate an indication for output, such as an alert for transmission, to a remote monitoring device, which may be external device 21 or a remote monitoring service, such as a cloud-based health monitoring service. System 8 may provide the alert to the remote monitoring device. System 8 may create such an indication and transmit such an indication for any tachyarrhythmia episodes associated with a fall, a slumping posture, or a change from an upright posture to a non-upright posture, whether or not the tachyarrhythmia episode was terminated by a shock (e.g., for ventricular tachyarrhythmia episodes). The indication may include one or more of the posture counts or signal over the course of the cardiac event, a length of time of the cardiac event, whether a fall, a slumping posture, or a change from an upright posture to a non-upright posture was detected, how far (e.g., in terms of time, such as x seconds) into the cardiac event was a fall or significant change in posture detected, a posture and/or activity after the cardiac event, etc.

Posture-modulated alert urgency for myocardial infarction detection is now discussed. System 8 may determine an elevated ST segment of an ECG. For example, system 8 may compare an ST segment of the ECG to a long term or short term running average of previous ST segments of the ECG to determine the ST segment is elevated. In some examples, system 8 may determine the ST segment is an elevated segment if the ST segment is a predetermined amount higher than the long term running average or the short term running average. In some examples, the predetermined amount may be on the order of 1 to 4 millivolts. In some examples, fall or posture detection may be disabled under normal operation to save on the limited battery power of IPD 16 and/or ICD 9. In such cases, upon determining the elevated ST segment, system 8 may enable fall and posture detection. For example, system 8 may begin to monitor changes in posture of the patient, after detecting an elevated ST segment via the accelerometer signal(s). System 8 may determine and store in memory any indication of a fall, a slumping posture, or a posture change from an upright posture to a non-upright posture. System 8 may generate for output an indication, such as an alert for transmission, to a remote monitoring device, such as external device 21 or a remote monitoring system, which may be a cloud-based remote health monitoring system. System 8 may transmit the indication to the remote monitoring device. The indication may include one or more of the elevated ST segments, an indication of the difference between the one or more elevated ST segments and the running average of the previous ST segments, information indicative of any fall, slump, or change in posture between an upright posture to a non-upright posture associated in time with the elevated ST segment, etc.

In some examples, system 8 may be programmed so as to enable fall, slump, and/or changes from an upright posture to a non-upright posture, without first requiring determination of an elevated ST segment. For example, such a feature may be useful for post-myocardial infarction patients. In such patients, the risk of sudden cardiac arrest may be higher than for others. As such, system 8 may be configured to immediately send an alert to an emergency medical service if system 8 determines a pause in heart beat, a fall, or a fall associated in time with a pause. As discussed above, system 8 may determine and store in memory any indication of a fall, a slump, or significant posture change from an upright posture to a non-upright posture associated with a pause. System 8 may create an indication for output, such as an alert, to a remote monitoring device. System 8 may transmit the alert to the remote monitoring device. The alert may include one or more of the elevated ST segment, an indication of the difference between the elevated ST segment and previous ST segments, information indicative of any fall, slumping posture, or any posture changes from an upright posture to a non-upright posture associated in time with the elevated ST segment, etc.

External device 21 may be configured to communicate with one or both of ICD 9 and IPD 16. In examples where external device 21 only communicates with one of subcutaneous ICD 9 and IPD 16, the non-communicative device may receive instructions from or transmit data to the device in communication with external device 21. In some examples, external device 21 comprises a handheld computing device, wearable device, tablet, laptop computer, computer workstation, networked computing device, or one or more servers. External device 21 may include a user interface that receives input from a user and provides information to the user, such as the indications and alerts discussed herein. In other examples, the user may also interact with external device 21 remotely via a networked computing device. The user may interact with external device 21 to communicate with IPD 16 and/or ICD 9. For example, the user may interact with external device 21 to send an interrogation request and retrieve therapy delivery data, update therapy parameters that define therapy, manage communication between IPD 16 and/or ICD 9, or perform any other activities with respect to IPD 16 and/or ICD 9. In some examples, the user is a clinician, such as a physician, technician, surgeon, electrophysiologist, or other healthcare professional. In some examples, the user may be patient 14, a family member of patient 14, a friend of patient 14, a caregiver of patient 14, or the like.

External device 21 may also allow the user to define how IPD 16 and/or ICD 9 senses electrical signals (e.g., ECGs), detects arrhythmias such as tachyarrhythmias, delivers therapy, and communicates with other devices of system 8. For example, external device 21 may be used to change tachyarrhythmia detection parameters. In another example, external device 21 may be used to manage therapy parameters that define therapies such as ATP therapy. Moreover, external device 21 may be used to alter communication protocols between IPD 16 and ICD 9. For example, external device 21 may instruct IPD 16 and/or ICD 9 to switch between one-way and two-way communication and/or change which of IPD 16 and/or ICD 9 are tasked with initial detection of arrhythmias.

External device 21 may communicate with IPD 16 and/or ICD 9 via wireless communication using any techniques known in the art. Examples of communication techniques may include, for example, proprietary and non-proprietary radiofrequency (RF) telemetry, but other techniques are also contemplated. In some examples, external device 21 may include a programming head that may be placed proximate to patient 14's body near the IPD 16 and/or ICD 9 implant site in order to improve the quality or security of communication between IPD 16 and/or ICD 9 and external device 21.

In some examples, IPD 16 and ICD 9 may engage in communication with each other and/or with external device 21 to facilitate the appropriate detection of cardiac events, detection of falls, slumping postures, or changes from an upright posture to a non-upright posture, delivery of cardiac therapy, such as anti-tachycardia therapy, and generation/transmission of indications regarding the cardiac event. Anti-tachycardia therapy may include ATP. The communication may include one-way communication in which one device is configured to transmit communication messages and the other device is configured to receive those messages. The communication may instead include two-way communication in which each device is configured to transmit and receive communication messages. Although the examples below describe detection of tachyarrhythmias and the delivery of ATP, IPD 16 and ICD 9 may be configured to communicate with each other and provide for the detection of other cardiac events, e.g., a pause in heartbeat, myocardial infarction, etc., and deliver alternative electrical stimulation therapies.

The leads and systems described herein may be used at least partially within the substernal space, e.g., within anterior mediastinum of patient, to provide a medical device system. An implanter (e.g., physician) may implant the distal portion of the lead intrathoracically using any of a number of implant tools, e.g., tunneling rod, sheath, or other tool that can traverse the diagrammatic attachments and form a tunnel in the substernal location. For example, the implanter may create an incision near the center of the torso of the patient, e.g., and introduce the implant tool into the substernal location via the incision. The implant tool is advanced from the incision superior along the posterior of the sternum in the substernal location. The distal end of lead 10 is introduced into tunnel via implant tool (e.g., via a sheath). As the distal end of lead 10 is advanced through the substernal tunnel, the distal end of lead 10 is relatively straight. The preformed or shaped undulating portion is flexible enough to be straightened out while routing the lead 10 through a sheath or other lumen or channel of the implant tool. Once the distal end of lead 10 is in place, the implant tool is withdrawn toward the incision and removed from the body of the patient while leaving lead 10 in place along the substernal path. As the implant tool is withdrawn, the distal end of lead 10 takes on its pre-formed undulating configuration. Thus, as the implant tool is withdrawn, the undulating configuration pushes electrodes 32a and 32b toward the left side of sternum compared to electrode segments 28a and 28b. As mentioned above, the implanter may align the electrodes 32a and 32b along the anterior median line (or midsternal line) or the left sternal lines (or left lateral sternal line).

Although system 8 is illustrated as including both extracardiovascular ICD system 6 and IPD 16, in some examples, a system may include only one of extracardiovascular ICD system 6 or IPD 16, and that component may perform the techniques described herein. In some examples, system 8 may include extracardiovascular ICD system 6 and IPD 16 and extracardiovascular ICD system 6 may, at some times, perform the techniques described herein in coordination with IPD 16 and, at other times, perform the techniques described herein without the use of IPD 16.

According to the techniques of this disclosure, system 8 may determine the occurrence of the cardiac event in patient 14. System 8 may determine, based at least in part on at least one accelerometer signal, at least one of a fall of patient 14, a slumping posture of patient 14, or a change from an upright posture to a non-upright posture of patient 14. System 8 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, system 8 may modulate a response to the cardiac event.

While the techniques of this disclosure may be discussed with respect to system 8 as a whole or with respect to specific devices of system 8, in some examples, the techniques of this disclosure may be performed by other devices, a single device, or any combination of devices. For example, processing circuitry of one or more devices may make the determinations based on accelerometer signal(s). For example, processing circuitry of one or more devices may cooperate to perform any of the techniques of this disclosure. In some examples, processing circuitry of external device 21 may perform the techniques of this disclosure or may cooperate with processing circuitry of one or more other devices to perform the techniques of this disclosure.

Although FIGS. 1A-1C are described in the context of an ICD 9 connected to lead 10 and IPD 16, the techniques may be applicable to other systems. For example, a medical device that includes a lead having a distal portion that is implanted above the sternum (or other extra-thoracic, subcutaneous location or intercostal location) instead of being implanted below the ribs and/or sternum. As another example, instead of an intracardiac pacing device, a pacing system may be implanted having a subcutaneous or submuscular pacemaker and one or more leads connected to and extending from the pacemaker into one or more chambers of the heart or attached to the outside of the heart to provide pacing therapy to the one or more chambers. As such, the example of FIGS. 1A-1C is illustrated for exemplary purposes only and should not be considered limiting of the techniques described herein.

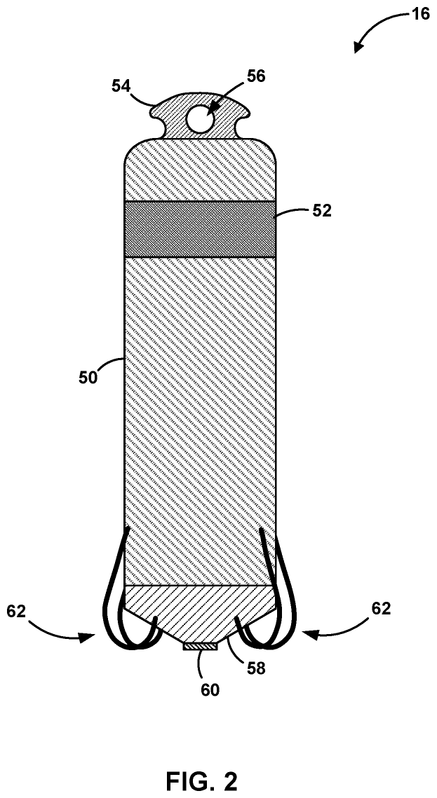

FIG. 2 is a conceptual drawing illustrating an example configuration of IPD 16 of the medical device system of FIGS. 1A-1C. As shown in FIG. 2, IPD 16 includes case 50, cap 58, electrode 60, electrode 52, fixation mechanisms 62, flange 54, and opening 56. Together, case 50 and cap 58 may be considered the housing of IPD 16. In this manner, case 50 and cap 58 may enclose and protect the various electrical components within IPD 16. Case 50 may enclose substantially all of the electrical components, and in some examples, one or more accelerometers, and cap 58 may seal case 50 and create the hermetically sealed housing of IPD 16. Although IPD 16 is generally described as including one or more electrodes, IPD 16 may typically include at least two electrodes (e.g., electrodes 52 and 60) to deliver an electrical signal (e.g., therapy such as ATP) and/or provide at least one sensing vector.

Electrodes 52 and 60 are carried on the housing created by case 50 and cap 58. In this manner, electrodes 52 and 60 may be considered leadless electrodes. In the example of FIG. 2, electrode 60 is disposed on the exterior surface of cap 58. Electrode 60 may be a circular electrode positioned to contact cardiac tissue upon implantation. Electrode 52 may be a ring or cylindrical electrode disposed on the exterior surface of case 50. Both case 50 and cap 58 may be electrically insulating. Electrode 60 may be used as a cathode and electrode 52 may be used as an anode, or vice versa, for delivering pacing stimulation therapy such as ATP. However, electrodes 52 and 60 may be used in any stimulation configuration. In addition, electrodes 52 and 60 may be used to detect intrinsic electrical signals from cardiac muscle. In other examples, IPD 16 may include three or more electrodes, where each electrode may deliver therapy and/or detect intrinsic signals. ATP delivered by IPD 16, as compared with alternative devices, may be considered to be “painless” to patient 14 or even undetectable by patient 14 since the electrical stimulation occurs very close to or at cardiac muscle and at relatively low energy levels.

Fixation mechanisms 62 may attach IPD 16 to cardiac tissue. Fixation mechanisms 62 may be active fixation tines, screws, clamps, adhesive members, or any other types of attaching a device to tissue. As shown in the example of FIG. 2, fixation mechanisms 62 may be constructed of a memory material that retains a preformed shape. During implantation, fixation mechanisms 62 may be flexed forward to pierce tissue and allowed to flex back towards case 50. In this manner, fixation mechanisms 62 may be embedded within the target tissue.

Flange 54 may be provided on one end of case 50 to enable tethering or extraction of IPD 16. For example, a suture or other device may be inserted around flange 54 and/or through opening 56 and attached to tissue. In this manner, flange 54 may provide a secondary attachment structure to tether or retain IPD 16 within heart 18 if fixation mechanisms 62 fail. Flange 54 and/or opening 56 may also be used to extract IPD 16 once the IPD needs to be explanted (or removed) from patient 14 if such action is deemed necessary.

The techniques described herein may generally be described with regard to a leadless pacing device such as IPD 16. IPD 16 may be an example of an anti-tachycardia pacing device (ATPD). However, alternative implantable medical devices may be used to perform the same or similar functions as IPD 16, e.g., delivering ATP or other therapy to heart 26 and, in some examples, communicate with ICD 9 and/or external device 21.

In another example, the ATPD may be configured to be implanted external to heart 26, e.g., near or attached to the epicardium of heart 26. An electrode carried by the housing of the ATPD may be placed in contact with the epicardium and/or one or more electrodes of leads coupled to the ATPD may be placed in contact with the epicardium at locations sufficient to provide therapy such as ATP (e.g., on external surfaces of the left and/or right ventricles). In any example, subcutaneous ICD 9 may communicate with one or more leadless or leaded devices implanted internal or external to heart 26.

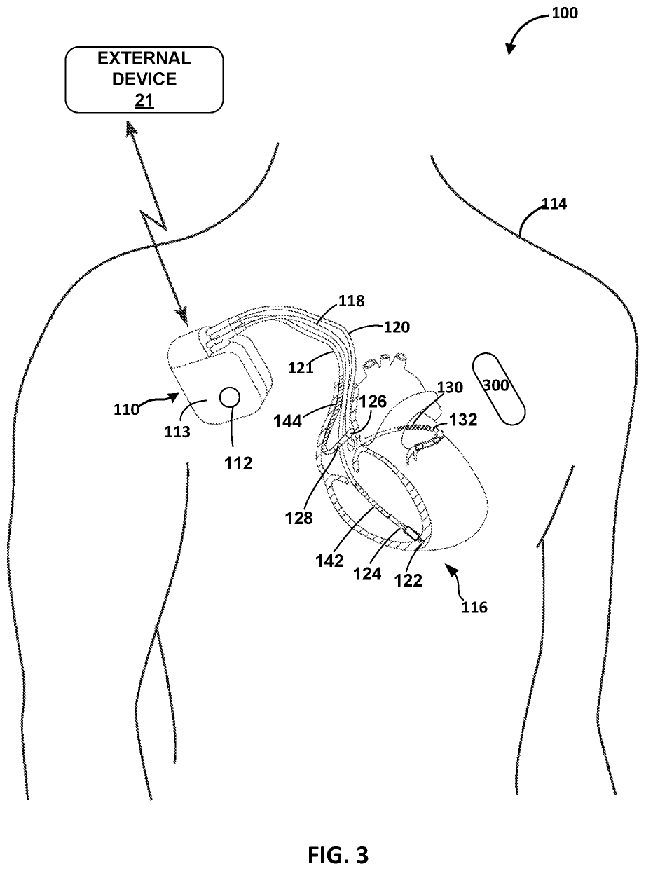

FIG. 3 is a schematic diagram of another example implantable medical device system 100 in conjunction with a patient 114. As illustrated in FIG. 3, a medical device system 100 for sensing cardiac events (e.g., P-waves, R-waves, QRS complexes, etc.) and detecting tachyarrhythmia episodes may include PCD 110, right ventricular lead 118, left ventricular lead 120, atrial lead 121, and insertable cardiac monitor (ICM) 300. In one example, PCD 110 may be embodied as an implantable cardioverter-defibrillator (ICD) capable of delivering pacing, cardioversion and defibrillation therapy to the heart 116 of a patient 114. Right ventricular lead 118, left ventricular lead 120 and atrial lead 121 are electrically coupled to PCD 110 and extend into the patient's heart 116 via a vein. Right ventricular lead 118 includes electrodes 122 and 124 shown positioned on the lead in the patient's right ventricle (RV) for sensing ventricular ECG signals and pacing in the RV. Left ventricular lead 120 includes electrode 130 positioned in the patient's left ventricle. Atrial lead 121 includes electrodes 126 and 128 positioned on the lead in the patient's right atrium (RA) for sensing atrial ECG signals and pacing in the RA.

Right ventricular lead 118 additionally carries high voltage coil electrodes 142 and 144 used to deliver cardioversion and defibrillation shock pulses. Each of right ventricle lead 118, left ventricular lead 120 and the atrial lead 121 may be used to acquire intracardiac ECG signals from the patient 114 and/or to deliver therapy in response to the acquired data. PCD 110 is shown as a dual chamber ICD, but in some examples, system 100 may be embodied as a multi-chamber system including a coronary sinus lead extending into the right atrium, through the coronary sinus and into a cardiac vein to position electrodes (e.g., electrode 130) along the left ventricle (LV) for sensing LV ECG signals and delivering pacing pulses to the LV.

Implantable medical device circuitry configured for performing the functions of PCD 110 described herein, and associated battery or batteries are housed within a sealed housing 113. In some examples, one or more accelerometers may also be housed within sealed housing 113. Housing 113 may be conductive so as to serve as an electrode for use as an indifferent electrode during pacing or sensing or as an active electrode during defibrillation. As such, housing 113 is also referred to herein as “housing electrode” 112.

ICM 300 may be a device for sensing extracardiac ECG signals. ICM 300 may be implanted within patient 14 and may communicate with PCD 110 and/or external device 21. ICM 300 may include a plurality of electrodes for sensing ECG signals.

EGM signal data acquired by PCD 110 may be transmitted to an external device 21. External device 21 may be embodied as a programmer, e.g. used in a clinic or hospital to communicate with PCD 110 via wireless telemetry. External device 21 may be coupled to a remote patient monitoring system, such as Carelink™, available from Medtronic plc, of Dublin, Ireland. External device 21 is used to program commands or operating parameters into PCD 110 for controlling IMD function and to interrogate PCD 110 to retrieve data, including device operational data as well as physiological data accumulated in IMD memory. Examples of communication techniques used by PCD 110 and external device 21 include low frequency or radiofrequency (RF) telemetry, which may be an RF link established via Bluetooth, WiFi, or MICS.

External device 21 may be configured to communicate with one or both of PCD 110 and ICM 300. In examples where external device 21 only communicates with one of PCD 110 and ICM 300, the non-communicative device may receive instructions from or transmit data to the device in communication with external device 21. In some examples, external device 21 comprises a handheld computing device, a wearable device, a tablet, a laptop computer, a computer workstation, a networked computing device, or one or more servers. External device 21 may include a user interface that receives input from a user and provides an indication or alert to the user as described herein. In other examples, the user may also interact with external device 21 remotely via a networked computing device. The user may interact with external device 21 to communicate with PCD 110 and/or ICM 300. For example, the user may interact with external device 21 to send an interrogation request and retrieve therapy delivery data, update therapy parameters that define therapy, manage communication between PCD 110 and/or ICM 300, or perform any other activities with respect to PCD 110 and/or ICM 300. Although the user is a physician, technician, surgeon, electrophysiologist, or other healthcare professional, the user may be patient 14, a family member of patient 14, a friend of patient 14, or a caregiver of patient 14, in some examples.

External device 21 may also allow the user to define how PCD 110 and/or ICM 300 senses electrical signals (e.g., ECGs), detects arrhythmias, such as tachyarrhythmias, determines a fall, a slumping posture, or a change from an upright posture to a non-upright posture of patient 14, delivers therapy, and communicates with other devices of system 100. For example, external device 21 may be used to change tachyarrhythmia detection parameters. In another example, external device 21 may be used to manage therapy parameters that define therapies such as ATP therapy. Moreover, external device 21 may be used to alter communication protocols between PCD 110 and ICM 300.

External device 21 may communicate with PCD 110 and/or ICM 300 via wireless communication using any techniques known in the art. Examples of communication techniques are described above with reference to FIG. 1A. In some examples, external device 21 may include a programming head that may be placed proximate to patient 14's body near the PCD 110 and/or ICM 300 implant site in order to improve the quality or security of communication between PCD 110 and/or ICM 300 and external device 21.

In some examples, PCD 110, ICM 300 and/or external device 21 may engage in communication to facilitate the appropriate detection of arrhythmias, the detection of a fall, a slumping posture, or a change from an upright posture to a non-upright posture, and/or delivery of anti-tachyarrhythmia. Anti-arrhythmia therapy may include ATP. The communication may include one-way communication in which one device is configured to transmit communication messages and another other device is configured to receive those messages. The communication may instead include two-way communication in which each device is configured to transmit and receive communication messages. Although the examples below describe detection of tachyarrhythmias and the delivery of ATP, PCD 110 and ICM 300 may be configured to communicate with each other to determine other cardiac events, such as a pause in heartbeat or myocardial infarction, and provide alternative cardiac therapies.

In some examples, system 100 may exclude ICM 300, and PCD 110 may deliver ATP therapy, sense evoked responses, and/or modify the ATP therapy based on the sensed evoked responses independently and/or in coordination with external device 21. In such an example, PCD 110 may use a plurality of pacing vectors to deliver ATP therapy to different locations in the heart without the need of ICM 300. In some examples, system 100 may exclude PCD 110.

According to the techniques of this disclosure, system 100 may be a medical device system that may determine the occurrence of the cardiac event in patient 14. System 100 may determine, based at least in part on at least one accelerometer signal, at least one of a fall of patient 14, a slumping posture of patient 14, or a change from an upright posture to a non-upright posture of patient 14. System 100 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, system 100 may modulate a response to the cardiac event.

While the techniques of this disclosure may be discussed with respect to system 100 as a whole or with respect to specific devices of system 100, in some examples, the techniques of this disclosure may be performed by other devices, a single device, or any combination of devices. For example, processing circuitry of one or more devices may make the determinations based on accelerometer signal(s). For example, processing circuitry of one or more devices may cooperate to perform any of the techniques of this disclosure. In some examples, processing circuitry of external device 21 may perform the techniques of this disclosure or may cooperate with processing circuitry of one or more other devices to perform the techniques of this disclosure.

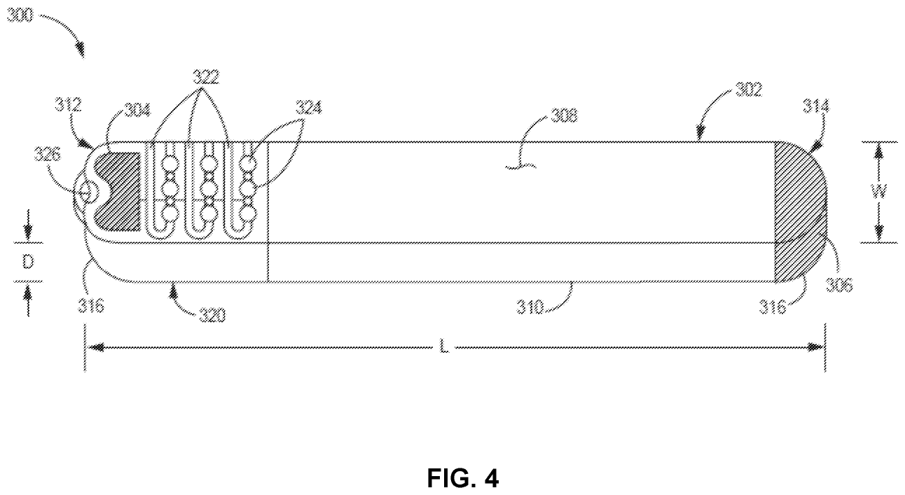

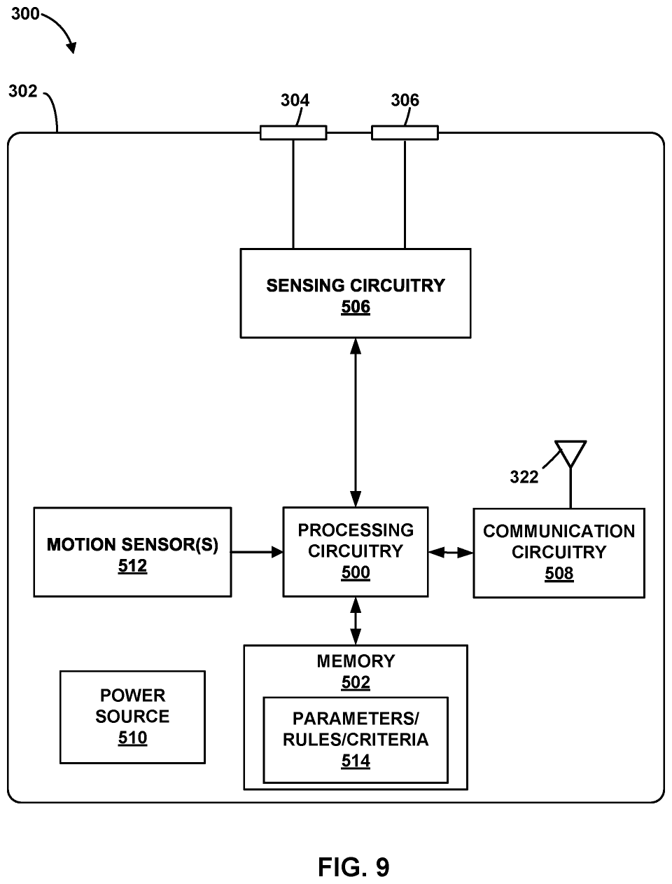

FIG. 4 is a conceptual drawing illustrating an example configuration of the insertable cardiac monitor (ICM) 300 of implantable medical device system 100 of FIG. 3. In the example shown in FIG. 4, ICM 300 may be embodied as a monitoring device having housing 302, proximal electrode 304 and distal electrode 306. Housing 302 may further comprise first major surface 308, second major surface 310, proximal end 312, and distal end 314. Housing 302 encloses electronic circuitry and a power source (shown in FIG. 9) located inside the ICM 300 and protects the circuitry and any accelerometers contained therein from body fluids. Electrical feedthroughs provide electrical connection of electrodes 304 and 306.

In the example shown in FIG. 4, ICM 300 is defined by a length L, a width W and thickness or depth D and is in the form of an elongated rectangular prism wherein the length L is much larger than the width W, which in turn is larger than the depth D. In one example, the geometry of the ICM 300—in particular a width W greater than the depth D—is selected to allow ICM 300 to be inserted under the skin of the patient using a minimally invasive procedure and to remain in the desired orientation during insertion. For example, the device shown in FIG. 4 includes radial asymmetries (notably, the rectangular shape) along the longitudinal axis that maintains the device in the proper orientation following insertion. For example, in one example the spacing between proximal electrode 304 and distal electrode 306 may range from 30 millimeters (mm) to 55 mm, 35 mm to 55 mm, and from 40 mm to 55 mm and may be any range or individual spacing from 25 mm to 60 mm. In addition, ICM 300 may have a length L that ranges from 30 mm to about 70 mm. In other examples, the length L may range from 40 mm to 60 mm, 45 mm to 60 mm and may be any length or range of lengths between about 30 mm and about 70 mm. In addition, the width W of major surface 308 may range from 3 mm to 10 mm and may be any single or range of widths between 3 mm and 10 mm. The thickness of depth D of ICM 300 may range from 2 mm to 9 mm. In other examples, the depth D of ICM 300 may range from 2 mm to 5 mm and may be any single or range of depths from 2 mm to 9 mm. In addition, ICM 300 according to an example of the present disclosure is has a geometry and size designed for ease of implant and patient comfort. Examples of ICM 300 described in this disclosure may have a volume of three cubic centimeters (cm) or less, 1.5 cubic cm or less or any volume between three and 1.5 cubic centimeters.

In the example shown in FIG. 4, once inserted within the patient, the first major surface 308 faces outward, toward the skin of the patient while the second major surface 310 is located opposite the first major surface 308. In addition, in the example shown in FIG. 4, proximal end 312 and distal end 314 are rounded to reduce discomfort and irritation to surrounding tissue once inserted under the skin of the patient.

Proximal electrode 304 and distal electrode 306 are used to sense cardiac signals, e.g., ECG signals, intra-thoracically or extra-thoracically, which may be sub-muscularly or subcutaneously. ECG signals may be stored in a memory of the ICM 300, and ECG data may be transmitted via integrated antenna 322 to another medical device, which may be another implantable device or an external device, such as PCD 110 or external device 21. In some example, electrodes 304 and 306 may additionally or alternatively be used for sensing any bio-potential signal of interest, which may be, for example, an electroencephalogram, ECG, or a nerve signal, from any implanted location.

In the example shown in FIG. 4, proximal electrode 304 is in close proximity to the proximal end 312 and distal electrode 306 is in close proximity to distal end 314. In this example, distal electrode 306 is not limited to a flattened, outward facing surface, but may extend from first major surface 308 around rounded edges 316 and/or end surface 318 and onto the second major surface 310 so that the electrode 306 has a three-dimensional curved configuration. In the example shown in FIG. 4, proximal electrode 304 is located on first major surface 308 and is substantially flat, outward facing. However, in other examples proximal electrode 304 may utilize the three-dimensional curved configuration of distal electrode 306, providing a three-dimensional proximal electrode (not shown in this example). Similarly, in other examples distal electrode 306 may utilize a substantially flat, outward facing electrode located on first major surface 308 similar to that shown with respect to proximal electrode 304. The various electrode configurations allow for configurations in which proximal electrode 304 and distal electrode 306 are located on both first major surface 308 and second major surface 310. In other configurations, such as that shown in FIG. 4, only one of proximal electrode 304 and distal electrode 306 is located on both major surfaces 308 and 310, and in still other configurations both proximal electrode 304 and distal electrode 306 are located on one of the first major surface 308 or the second major surface 310 (i.e., proximal electrode 304 located on first major surface 308 while distal electrode 306 is located on second major surface 310). In another example, ICM 300 may include electrodes on both major surface 308 and 310 at or near the proximal and distal ends of the device, such that a total of four electrodes are included on ICM 300. Electrodes 304 and 306 may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 4, proximal end 312 includes a header assembly 320 that includes one or more of proximal electrode 304, integrated antenna 322, anti-migration projections 324, and/or suture hole 326. Integrated antenna 322 is located on the same major surface (i.e., first major surface 308) as proximal electrode 304 and is also included as part of header assembly 320. Integrated antenna 322 allows ICM 300 to transmit and/or receive data. In other examples, integrated antenna 322 may be formed on the opposite major surface as proximal electrode 304, or may be incorporated within the housing 302 of ICM 300. In the example shown in FIG. 4, anti-migration projections 324 are located adjacent to integrated antenna 322 and protrude away from first major surface 308 to prevent longitudinal movement of the device. In the example shown in FIG. 4, anti-migration projections 324 includes a plurality (e.g., nine) small bumps or protrusions extending away from first major surface 308. As discussed above, in other examples anti-migration projections 324 may be located on the opposite major surface as proximal electrode 304 and/or integrated antenna 322. In addition, in the example shown in FIG. 4 header assembly 320 includes suture hole 326, which provides another means of securing ICM 300 to the patient to prevent movement following insert. In the example shown, suture hole 326 is located adjacent to proximal electrode 304. In one example, header assembly 320 is a molded header assembly made from a polymeric or plastic material, which may be integrated or separable from the main portion of ICM 300.

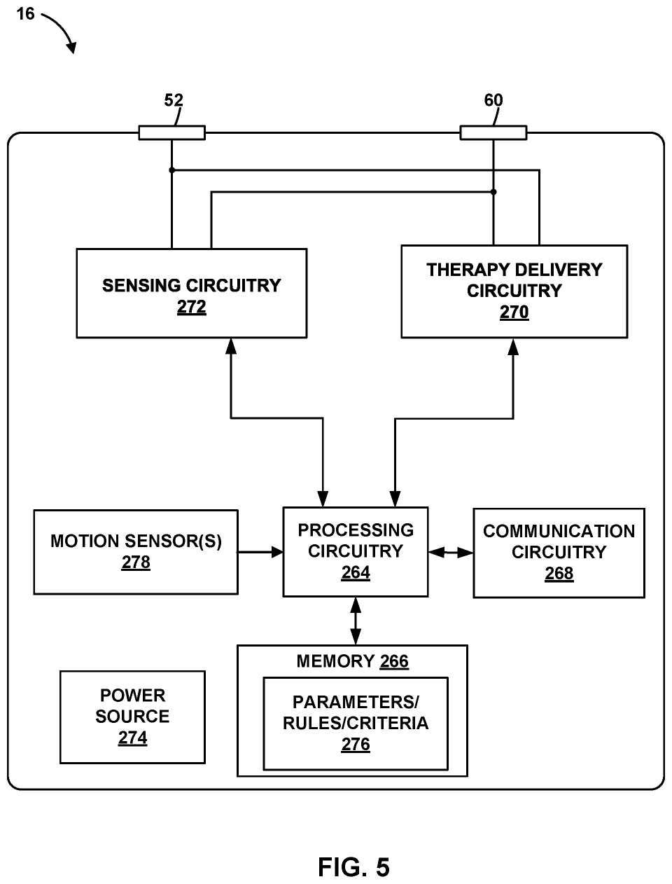

FIG. 5 is a functional block diagram illustrating an example configuration of IPD 16 of FIGS. 1A-1C and 2. In the illustrated example, IPD 16 includes processing circuitry 264, memory 226, therapy delivery circuitry 270, sensing circuitry 272, communication circuitry 268, motion sensor(s) 278 (which may include one or more accelerometers), and power source 274. The electronic components may receive power from a power source 274, which may be a rechargeable or non-rechargeable battery. In other examples, IPD 16 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitries may be performed by separate hardware or software components, or integrated within common or separate hardware or software components.

Memory 226 includes computer-readable instructions that, when executed by processing circuitry 264, cause IPD 16 and processing circuitry 264 to perform various functions attributed to IPD 16 and processing circuitry 264 herein (e.g., determining a cardiac event, determining a fall, a slumping posture, or a change from an upright posture to a non-upright posture, delivering therapy, and/or generating an indication for output). Memory 226 may include any volatile, non-volatile, magnetic, optical, or electrical media, such as a random access memory (RAM), ferroelectric RAM (FRAM), read-only memory (ROM), non-volatile RAM (NVRAM), electrically-erasable programmable ROM (EEPROM), flash memory, or any other digital or analog media.

Processing circuitry 264 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 264 may include multiple components, such as any combination of one or more microprocessors, one or more controllers, one or more DSPs, one or more ASICs, or one or more FPGAs, as well as other discrete or integrated logic circuitry. The functions attributed to processing circuitry 264 herein may be embodied as software, firmware, hardware or any combination thereof.

Therapy delivery circuitry 270 is electrically coupled to electrodes 52 and 60 carried on the housing of IPD 16. Therapy delivery circuitry 270 may include one or more pulse generators, capacitors, and/or other components capable of generating and/or storing energy to deliver as pacing therapy. In the illustrated example, therapy delivery circuitry 270 is configured to generate and deliver electrical stimulation therapy to heart 26. For example, therapy delivery circuitry 270 may deliver the electrical stimulation therapy to a portion of cardiac muscle within heart 26 via electrodes 52 and 60. In some examples, therapy delivery circuitry 270 may deliver pacing stimulation, e.g., ATP therapy, in the form of voltage or current electrical pulses. In other examples, therapy delivery circuitry 270 may deliver stimulation in the form of other signals, such as sine waves, square waves, or other substantially continuous time signals.

Motion sensor(s) 278 may include a three-axis accelerometer, one or more single-axis accelerometers, or the like. Motion sensor(s) 278 may be configured to generate one or more signals that may be indicative of a posture of patient 14, for example, an upright posture, a slumping posture, a non-upright posture, or the like. Additionally, or alternatively, motion sensor(s) 278 may be configured to generate a spike in one or more signals that may be indicative of a fall. For example, the spike may correspond to an acceleration of patient 14 towards the ground when falling, or a spike may correspond to an impact with the ground caused by falling.

Processing circuitry 264 may monitor the one or more signals of motion sensor(s) 278 to determine whether patient 14 has fallen, slumping, or otherwise changed from an upright posture to a non-upright posture. In some examples, processing circuitry 264 may modulate at least one of a therapy delivery, e.g., of ATP, or an alert in response to a determination that patient 14 has fallen, slumping, or otherwise changed from an upright posture to a non-upright posture.

In some examples, upon determining that patient 14 has fallen, slumping, or otherwise changed from an upright posture to a non-upright posture, processing circuitry 264 may relax or override rules of a cardiac rhythm discrimination algorithm and control therapy delivery circuitry to deliver a shock or other therapy to heart 26 of patient 14. Such rules may be stored in parameters/rules/criteria 276 of memory 226. In some examples, upon determining that patient 14 has fallen, slumping, or otherwise changed from an upright posture to a non-upright posture, processing circuitry 264 may relax onset detection criteria, which may be stored in parameters/rules/criteria 276, to make it more likely that processing circuitry 264 may detect and treat a cardiac event.

Processing circuitry 264 controls therapy delivery circuitry 270 to deliver cardiac pacing therapy or other therapy to heart 26 according to parameters, which may be stored in parameters/rules/criteria 276. For example, processing circuitry 264 may control therapy delivery circuitry 270 to deliver pacing pulses with the amplitudes, pulse widths, frequency, or electrode polarities specified by the therapy parameters, including intervals that define under what conditions and when pacing pulses should be delivered. In this manner, therapy delivery circuitry 270 may deliver pacing pulses (e.g., ATP pulses) to heart 26 via electrodes 52 and 60. Although IPD 16 may only include two electrodes, e.g., electrodes 52 and 60, IPD 16 may utilize three or more electrodes in other examples. IPD 16 may use any combination of electrodes to deliver therapy and/or detect electrical signals from patient 14.

Processing circuitry 264 may control therapy delivery circuitry 270 to deliver pacing pulses for ATP therapy according to ATP parameters in parameters/rules/criteria 276 stored in memory 266. ATP therapy parameters may include pulse intervals, pulse width, current and/or voltage amplitudes, and durations for each pacing mode. In some examples, ATP parameters may include a plurality of ranges of propagation times and associated numbers of pulses of an ATP train, a formula that may be applied to a first propagation time, a linear equation that may be applied to a first propagation time, a continuous function that may be applied to a first propagation time, or a look-up table that may include first propagation times and associated number of pulses of an ATP train. For example, the pulse interval may be based on a fraction of the detected ventricular tachycardia cycle length and be between approximately 150 milliseconds and 500 milliseconds (e.g., between approximately 2.0 hertz and 7.0 hertz), and the pulse width may be between approximately 0.5 milliseconds and 2.0 milliseconds. The amplitude of each pacing pulse may be between approximately 2.0 volts and 10.0 volts. In some examples, the pulse amplitude may be approximately 6.0 V and the pulse width may be approximately 1.5 milliseconds; another example may include pulse amplitudes of approximately 5.0 V and pulse widths of approximately 1.0 milliseconds.

Each train of pulses during ATP may last for a duration of between approximately 70 milliseconds to approximately 15 seconds or be defined as a specific number of pulses.

Processing circuitry 264 controls therapy delivery circuitry 270 to generate and deliver pacing pulses with any of a number of shapes, amplitudes, pulse widths, or other characteristic to capture the heart. For example, the pacing pulses may be monophasic, biphasic, or multi-phasic (e.g., more than two phases). The pacing thresholds of the heart when delivering pacing pulses may depend upon a number of factors, including location, type, size, orientation, and/or spacing of IPD 16 and/or electrodes 52 and/or 60, physical abnormalities of the heart (e.g., pericardial adhesions or myocardial infarctions), or other factor(s).

In examples in which IPD 16 includes more than two electrodes, therapy delivery circuitry 270 may include a switch and processing circuitry 264 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes.

Sensing circuitry 272 is electrically connected to and monitors signals from some or all of electrodes 52 and 60 in order to monitor electrical activity of heart 26, impedance, or other electrical phenomenon. Sensing may be done to determine heart rates or heart rate variability, or to detect arrhythmias (e.g., tachyarrhythmias or bradycardia) or other electrical signals. Sensing circuitry 272 may also include a switch to select which of the available electrodes (or electrode polarity) are used to sense the heart activity, depending upon which electrode combination, or electrode vector, is used in the current sensing configuration. In examples with several electrodes, processing circuitry 264 may select the electrodes that function as sense electrodes, i.e., select the sensing configuration, via the switch circuitry within sensing circuitry 272. Sensing circuitry 272 may include one or more detection channels, each of which may be coupled to a selected electrode configuration for detection of cardiac signals via that electrode configuration. Some detection channels may be configured to detect cardiac events, such as fiducial points, QRS complexes, P- or R-waves, and provide indications of the occurrences of such events to processing circuitry 264, e.g., as described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Processing circuitry 264 may control the functionality of sensing circuitry 272 by providing signals via a data/address bus.

The components of sensing circuitry 272 may be analog components, digital components or a combination thereof. Sensing circuitry 272 may, for example, include one or more sense amplifiers, filters, rectifiers, threshold detectors, analog-to-digital converters (ADCs) or the like. Sensing circuitry 272 may convert the sensed signals to digital form and provide the digital signals to processing circuitry 264 for processing or analysis. For example, sensing circuitry 272 may amplify signals from the sensing electrodes and convert the amplified signals to multi-bit digital signals by an ADC. Sensing circuitry 272 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 264.

Sensing circuitry 272 and/or processing circuitry 264 may also include circuitry for measuring the capture threshold for the delivery of pacing pulses via electrodes 52 and 60. The capture threshold may indicate the voltage and pulse width necessary to induce depolarization of the surrounding cardiac muscle. For example, processing circuitry 264 may periodically control therapy delivery circuitry 270 to modify the amplitude of pacing pulses delivered to patient 14, and sensing circuitry 272 and/or processing circuitry 264 may detect whether the surrounding cardiac tissue depolarized in response to the pacing pulses, i.e., detected whether there was an evoked response to the pacing pulse. Processing circuitry 264 may determine the capture threshold based on the amplitude where loss of capture occurred.

Processing circuitry 264 may process the signals from sensing circuitry 272 to monitor electrical activity of the heart of the patient. Processing circuitry 264 may store signals obtained by sensing circuitry 272 as well as any generated ECG waveforms, marker channel data or other data derived based on the sensed signals in memory 266. Processing circuitry 264 may analyze the ECG waveforms and/or marker channel data to detect cardiac events (e.g., tachycardia). In response to detecting the cardiac event, processing circuitry 264 may control therapy delivery circuitry 270 to deliver the desired therapy to treat the cardiac event, e.g., ATP therapy.

In examples in which IPD 16 includes more than two electrodes, therapy delivery circuitry 270 may include a switch and processing circuitry 264 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes. Processing circuitry 264 may select the electrodes to function as signal electrodes, or the signal vector, via the switch circuitry within therapy delivery circuitry 270. In some instances, the same switch circuitry may be used by both therapy delivery circuitry 270 and sensing circuitry 272. In other instances, each of sensing circuitry 272 and therapy delivery circuitry 270 may have separate switch circuitry.

Processing circuitry 264 may include a timing and control circuitry, which may be embodied as hardware, firmware, software, or any combination thereof. The timing and control circuitry may comprise a dedicated hardware circuit, such as an ASIC, separate from other processing circuitry 264 components, such as a microprocessor, or a software module executed by a component of processing circuitry 264, which may be a microprocessor or ASIC. The timing and control circuitry may implement programmable counters. If IPD 16 is configured to generate and deliver pacing pulses to heart 26, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control circuitry within processing circuitry 264 may include atrial and ventricular pacing escape intervals, refractory periods during which sensed P-waves and R-waves are ineffective to restart timing of the escape intervals, and the pulse widths of the pacing pulses. As another example, the timing and control circuitry may withhold sensing from one or more channels of sensing circuitry 272 for a time interval during and after delivery of electrical stimulation to heart 26. The durations of these intervals may be determined by processing circuitry 264 in response to stored data in memory 226. The timing and control circuitry of processing circuitry 264 may also determine the amplitude of the cardiac pacing pulses.

Interval counters implemented by the timing and control circuitry of processing circuitry 264 may be reset upon sensing of R-waves and P-waves with detection channels of sensing circuitry 272. In examples in which IPD 16 provides pacing, therapy delivery circuitry 270 may include pacer output circuits that are coupled to electrodes 52 and 60, for example, appropriate for delivery of a bipolar or unipolar pacing pulse to one of the chambers of heart 26. In such examples, processing circuitry 264 may reset the interval counters upon the generation of pacing pulses by therapy delivery circuitry 270, and thereby control the basic timing of cardiac pacing functions, including ATP or post-shock pacing.

The value of the count present in the interval counters when reset by sensed R-waves and P-waves may be used by processing circuitry 264 to measure the durations of R-R intervals, P-P intervals, P-R intervals and R-P intervals, which are measurements that may be stored in memory 266. Processing circuitry 264 may use the count in the interval counters to detect a tachyarrhythmia event, such as atrial fibrillation, atrial tachycardia, ventricular fibrillation, or ventricular tachycardia. These intervals may also be used to detect the overall heart rate, ventricular contraction rate, and heart rate variability. A portion of memory 266 may be configured as a plurality of recirculating buffers, capable of holding series of measured intervals, which may be analyzed by processing circuitry 264 in response to the occurrence of a pace or sense interrupt to determine whether the patient's heart 26 is presently exhibiting atrial or ventricular tachyarrhythmia. In some examples, an arrhythmia detection method may include any suitable tachyarrhythmia detection algorithms.

In some examples, processing circuitry 264 may determine that tachyarrhythmia has occurred by identification of shortened R-R (or P-P) interval lengths. Generally, processing circuitry 264 detects tachycardia when the interval length falls below 220 milliseconds and fibrillation when the interval length falls below 180 milliseconds. In other examples, processing circuitry 264 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 266. This interval length may need to be detected for a certain number of consecutive cycles, for a certain percentage of cycles within a running window, or a running average for a certain number of cardiac cycles, as examples. In other examples, additional physiological parameters may be used to detect an arrhythmia. For example, processing circuitry 264 may analyze one or more morphology measurements, impedances, or any other physiological measurements to determine that patient 14 is experiencing a tachyarrhythmia.

In the event that an ATP regimen is desired, timing intervals for controlling the generation of ATP therapies by therapy deliver circuitry 270 may be loaded by processing circuitry 264 into the timing and control circuitry based on ATP parameters 276 to control the operation of the escape interval counters therein and to define refractory periods during which detection of R-waves and P-waves is ineffective to restart the escape interval counters for the ATP. An ATP regimen may be desired if processing circuitry 264 detects an atrial or ventricular tachyarrhythmia based on signals from sensing circuitry 272, and/or receives a command from another device or system, such as ICD 9, as examples.

In addition to detecting and identifying specific types of cardiac rhythms, sensing circuitry 272 may also sample the detected intrinsic signals to generate an ECG or other time-based indication of cardiac events. Processing circuitry 264 may also be able to coordinate the delivery of pacing pulses from different IPDs implanted in different chambers of heart 26, such as an IPD implanted in atrium and/or an IPD implanted in left ventricle. For example, processing circuitry 264 may identify delivered pulses from other IPDs via sensing circuitry 272 and update pulse timing to accomplish a selected pacing regimen. This detection may be on a pulse-to-pulse or beat-to-beat basis, or on a less frequent basis to make slight modifications to pulse rate over time. In other examples, IPDs may communicate with each other via communication circuitry 268 and/or instructions over a carrier wave (such as a stimulation waveform). In this manner, ATP pacing may be coordinated from multiple IPDs.

IPD 16 may deliver ATP therapy using electrodes 52 and 60 and therapy delivery circuitry 270 and may sense a local evoked response using the electrodes 52 and 60 and sensing circuitry 272 to sense at a location that is at or near the location of the delivery of the ATP therapy. Another device, such as ICD 9 may sense a global evoked response to the ATP pacing delivered by IPD 16 by sensing at a location that is a substantial distance from the location of the delivery of the ATP therapy. In other examples, the same device, using different electrode vectors, may be used to sense both local and global evoked responses to delivered ATP therapy delivered by the device. The evoked responses may be detected via the hardware of sensing circuitry 272 similar to R-wave, e.g., using a sense amplifier to detect amplitude above a threshold shortly after delivery of a pacing pulse, and/or may be by detected by processing circuitry 264 determining a spike by signal processing a digitized version of ECG signals from electrodes 52 and 60.

Memory 266 may be configured to store a variety of operational parameters, therapy parameters, including ATP therapy parameters, sensed and detected data, and any other information related to the therapy and treatment of patient 14. In the example of FIG. 5, memory 266 may store sensed ECGs, detected arrhythmias, detected falls, slumping postures, or changes from upright postures to non-upright postures, communications from ICD 9 and/or external device 21, and therapy parameters that define ATP therapy (which may be stored in parameters/rules/criteria 276). In other examples, memory 266 may act as a temporary buffer for storing data until it can be uploaded to ICD 9, another implanted device, or external device 21.

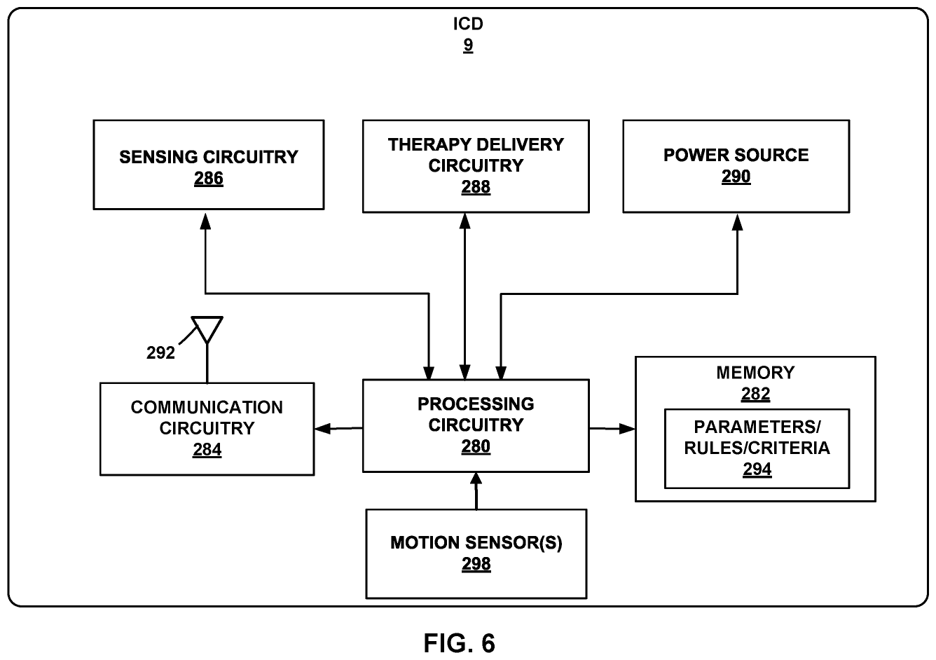

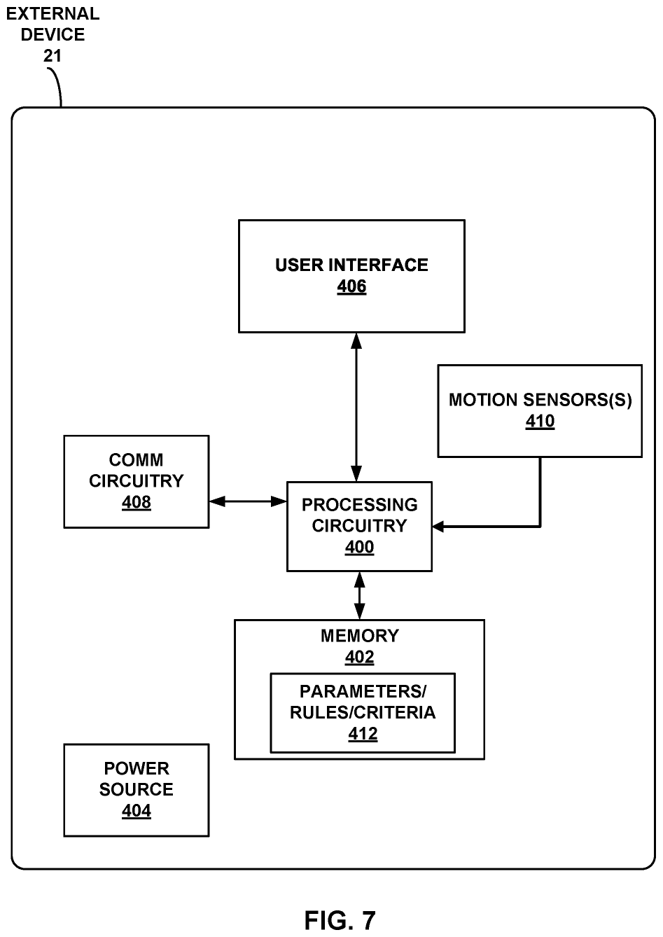

Communication circuitry 268 includes any suitable hardware, firmware, software or any combination thereof for communicating with another device, such as external device 21 (FIGS. 1A-1C and 7), ICD 9 (FIGS. 1A-1C and 6), a clinician programmer, a patient monitoring device, or the like. For example, communication circuitry 284 may include appropriate modulation, demodulation, frequency conversion, filtering, and amplifier components for transmission and reception of data. Under the control of processing circuitry 264, communication circuitry 268 may receive downlink telemetry from and send uplink telemetry to external device 21 with the aid of an antenna, which may be internal and/or external. Processing circuitry 264 may provide the data to be uplinked to external device 21 and the control signals for the telemetry circuit within communication circuitry 268, e.g., via an address/data bus. In some examples, communication circuitry 268 may provide received data to processing circuitry 264 via a multiplexer.

In some examples, IPD 16 may signal external device 21 to further communicate with and pass the alert through a network such as the Medtronic CareLink® Network developed by Medtronic plc., of Dublin, Ireland, or some other network linking patient 14 to a clinician. IPD 16 may spontaneously transmit information to the network or in response to an interrogation request from a user.

Power source 274 may be any type of device that is configured to hold a charge to operate the circuitry of IPD 16. Power source 274 may be provided as a rechargeable or non-rechargeable battery. In other example, power source 274 may incorporate an energy scavenging system that stores electrical energy from movement of IPD 16 within patient 14.

The various circuitry of IPD 16 may include any one or more processors, controllers, digital signal processors (DSPs), application specific integrated circuits (ASICs), field programmable gate arrays (FPGAs), or equivalent discrete or integrated circuitry, including analog circuitry, digital circuitry, or logic circuitry.

According to the techniques of this disclosure, processing circuitry 264 may determine the occurrence of the cardiac event in patient 14. Processing circuitry 264 may determine, based at least in part on at least one accelerometer signal, at least one of a fall of patient 14, a slumping posture of patient 14, or a change from an upright posture to a non-upright posture of patient 14. Processing circuitry 264 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, processing circuitry 264 may modulate a response to the cardiac event.

FIG. 6 is a functional block diagram illustrating an example configuration of ICD 9 of the implantable medical device system of FIGS. 1A-1C. In the illustrated example, ICD 9 includes processing circuitry 280, sensing circuitry 286, therapy delivery circuitry 288, communication circuitry 284, motion sensor(s) 298 (which may include one or more accelerometers), and memory 282. The electronic components may receive power from a power source 290, which may be a rechargeable or non-rechargeable battery. In other examples, ICD 9 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components. FIG. 6 will be described in the context of ICD 9 being coupled to lead 10 for exemplary purposes only. However, ICD 9 may be coupled to other leads, and thus other electrodes.

Memory 282 includes computer-readable instructions that, when executed by processing circuitry 280, cause ICD 9 and processing circuitry 280 to perform various functions attributed to ICD 9 and processing circuitry 280 herein (e.g., detecting a cardiac event, determining a fall, a slumping posture, or a change from an upright posture to a non-upright posture, delivering cardiac therapy, such as anti-tachycardia pacing, and/or generating/transmitting an indication regarding the cardiac event). Memory 282 may include any volatile, non-volatile, magnetic, optical, or electrical media, such as RAM, ROM, FRAM, NVRAM, EEPROM, flash memory, or any other digital or analog media.

Sensing circuitry 286 is electrically coupled to some or all of electrode 28 (or separately to segments 28a and/or 28b) and 32a and 32b via the conductors of lead 10 (shown in FIGS. 1A-1C) and one or more electrical feedthroughs, or to the housing electrode via conductors internal to the housing of ICD 9. Sensing circuitry 286 is configured to obtain signals sensed via one or more combinations of electrode 28 (or separately to segments 28a and/or 28b), 32a, 32b and the housing electrode of ICD 9 and process the obtained signals.

The components of sensing circuitry 286 may be analog components, digital components or a combination thereof. Sensing circuitry 286 may, for example, include one or more sense amplifiers, filters, rectifiers, threshold detectors, ADCs, or the like. Sensing circuitry 286 may convert the sensed signals to digital form and provide the digital signals to processing circuitry 280 for processing or analysis. For example, sensing circuitry 286 may amplify signals from the sensing electrodes and convert the amplified signals to multi-bit digital signals by an ADC. Sensing circuitry 286 may also compare processed signals to a threshold to detect the existence of fiducial points, QRS complexes, and atrial or ventricular depolarizations (e.g., P- or R-waves) and indicate the existence of the atrial depolarization (e.g., P-waves) or ventricular depolarizations (e.g., R-waves) to processing circuitry 280.

Processing circuitry 280 may include any one or more of a microprocessor, a controller, a DSP, an ASIC, an FPGA, or equivalent discrete or analog logic circuitry. In some examples, processing circuitry 280 may include multiple components, such as any combination of one or more microprocessors, one or more controllers, one or more DSPs, one or more ASICs, or one or more FPGAs, as well as other discrete or integrated logic circuitry. The functions attributed to processing circuitry 280 herein may be embodied as software, firmware, hardware or any combination thereof.

Processing circuitry 280 may process the signals from sensing circuitry 286 to monitor electrical activity of the heart of the patient. Processing circuitry 280 may store signals obtained by sensing circuitry 286 as well as any generated ECG waveforms, marker channel data, accelerometer data, or other data derived based on the sensed signals in memory 282. Processing circuitry 280 may analyze the ECG waveforms and/or marker channel data to detect cardiac events (e.g., tachycardia). In response to detecting the cardiac event, processing circuitry 280 may control therapy delivery circuitry 288 to deliver the desired therapy to treat the cardiac event, e.g., ATP therapy.

Therapy delivery circuitry 288 is configured to generate and deliver electrical stimulation therapy to the heart. Therapy delivery circuitry 288 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 288 may include a first set of components configured to provide pacing therapy and a second set of components configured to provide defibrillation therapy. In other instances, therapy delivery circuitry 288 may utilize the same set of components to provide both pacing and defibrillation therapy. In still other instances, therapy delivery circuitry 288 may share some of the defibrillation and pacing therapy components while using other components solely for defibrillation or pacing. In some examples, therapy delivery circuitry 288 may deliver pacing stimulation, e.g., ATP therapy, in the form of voltage or current electrical pulses. In other examples, therapy delivery circuitry 288 may deliver stimulation in the form of other signals, such as sine waves, square waves, or other substantially continuous time signals.

Motion sensor(s) 298 may include a three-axis accelerometer, one or more single-axis accelerometers, or the like. Motion sensor(s) 298 may be configured to generate one or more signals that may be indicative of a posture of patient 14, for example, an upright posture, a slumping posture, a non-upright posture, or the like. Additionally, or alternatively, motion sensor(s) 298 may be configured to generate a spike in one or more signals that may be indicative of a fall. For example, the spike may correspond to an acceleration of patient 14 towards the ground when falling, or a spike may correspond to an impact with the ground caused by falling.

Processing circuitry 280 may monitor the one or more signals of motion sensor(s) 298 to determine whether patient 14 has fallen, slumping, or otherwise changed from an upright posture to a non-upright posture. In some examples, processing circuitry 280 may modulate at least one of a therapy delivery or an alert in response to a determination that patient 14 has fallen, slumping, or otherwise changed from an upright posture to a non-upright posture.

In some examples, upon determining that patient 14 has fallen, slumping, or otherwise changed from an upright posture to a non-upright posture, processing circuitry 280 may relax or override rules of a cardiac rhythm discrimination algorithm and control therapy delivery circuitry to deliver a shock or other therapy to heart 26 of patient 14. Such rules may be stored in parameters/rules/criteria 294 of memory 282. In some examples, upon determining that patient 14 has fallen, slumping, or otherwise changed from an upright posture to a non-upright posture, processing circuitry 280 may relax onset detection criteria, which may be stored in parameters/rules/criteria 294, to make it more likely that processing circuitry 280 may detect and treat a cardiac event.

Processing circuitry 280 may control therapy delivery circuitry 288 to deliver the generated therapy to the heart via one or more combinations of electrode 28 (or separately to segments 28a and/or 28b), 32a, and 32b of lead 10 and the housing electrode of ICD 9 according to one or more therapy programs, which may be stored in memory 282. In instances in which processing circuitry 280 is coupled to a different lead, other electrodes may be utilized. Processing circuitry 280 controls therapy delivery circuitry 288 to generate electrical stimulation therapy with the amplitudes, pulse widths, timing, frequencies, electrode combinations or electrode configurations specified by stored therapy programs.

Processing circuitry 280 controls therapy delivery circuitry 288 to deliver cardiac pacing therapy to heart 26 according to parameters, which may be stored in parameters/rules/criteria 294 in memory 282. For example, processing circuitry 280 may control therapy delivery circuitry 288 to deliver pacing pulses with the amplitudes, pulse widths, frequency, or electrode polarities specified by the therapy parameters, including intervals that define under what conditions and when pacing pulses should be delivered.

Processing circuitry 280 may control therapy delivery circuitry 288 to deliver ATP therapy based on ATP parameters stored in parameters/rules/criteria 294 and may modify the ATP therapy by modifying the ATP parameters. ATP therapy parameters may include pulse intervals, pulse width, current and/or voltage amplitudes, and durations for each pacing mode. In some examples, ATP parameters may include a plurality of ranges of propagation times and associated numbers of pulses of an ATP train, a formula that may be applied to a first propagation time, a linear equation that may be applied to a first propagation time, a continuous function that may be applied to a first propagation time, or a look-up table that may include first propagation times and associated numbers of pulses of an ATP train. For example, the pulse interval may be based on a fraction of the detected ventricular tachycardia cycle length and be between approximately 150 milliseconds and 500 milliseconds (e.g., between approximately 2.0 hertz and 7.0 hertz), and the pulse width may be between approximately 0.5 milliseconds and 2.0 milliseconds. The amplitude of each pacing pulse may be between approximately 2.0 volts and 10.0 volts. In some examples, the pulse amplitude may be approximately 6.0 V and the pulse width may be approximately 1.5 milliseconds; another example may include pulse amplitudes of approximately 5.0 V and pulse widths of approximately 1.0 milliseconds.

Each pulse, or burst of pulses, may include a ramp up in amplitude or in pulse rate. In addition, trains of pulses in successive ATP periods may be delivered at increasing pulse rate in an attempt to capture the heart and terminate the tachycardia.

Therapy delivery circuitry 288 may include switch circuitry to select which of the available electrodes are used to deliver the therapy. The switch circuitry may include a switch array, switch matrix, multiplexer, or any other type of switching device suitable to selectively couple electrodes to therapy delivery circuitry 288. Processing circuitry 280 may select the electrodes to function as signal electrodes, or the signal vector, via the switch circuitry within therapy delivery circuitry 288. In instances in which defibrillation segments 28a and 28b are each coupled to separate conductors, processing circuitry 280 may be configured to selectively couples therapy delivery circuitry 288 to either one of segments 28a and 28b individually or couple to both of the segments 28a and 28b concurrently. In some instances, the same switch circuitry may be used by both therapy delivery circuitry 288 and sensing circuitry 286. In other instances, each of sensing circuitry 286 and therapy delivery circuitry 288 may have separate switch circuitry.

According to the techniques of this disclosure, processing circuitry 280 may determine the occurrence of the cardiac event in patient 14. Processing circuitry 280 may determine, based at least in part on at least one accelerometer signal, at least one of a fall of patient 14, a slumping posture of patient 14, or a change from an upright posture to a non-upright posture of patient 14. Processing circuitry 280 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, processing circuitry 280 may modulate a response to the cardiac event.

In one example, therapy delivery circuitry 288 may deliver pacing via an electrode vector that includes one or both defibrillation electrode segments 28a and 28b. The electrode vector used for pacing may be segment 28a as an anode (or cathode) and one of segment 28b, or electrodes 32a or 32b, or the housing of ICD 9 as the cathode (or anode) or segment 28b as an anode (or cathode) and one of segment 28b, or electrodes 32a or 32b, or the housing of ICD 9 as the cathode (or anode). In some examples, electrode 52 and/or electrode 60 of IPD 16 may be used as an anode or cathode and ICD 9 may communicate with 16 via communication circuitry 284 and/or external device may communicate with ICD 9 and IPD 16 to coordinate the use of electrodes of both ICD 9 and IPD 16.

Processing circuitry 280 controls therapy delivery circuitry 288 to generate and deliver pacing pulses with any of a number of shapes, amplitudes, pulse widths, or other characteristic to capture the heart. For example, the pacing pulses may be monophasic, biphasic, or multi-phasic (e.g., more than two phases). The pacing thresholds of the heart when delivering pacing pulses from the substernal space, e.g., from electrodes 32a, 32b and/or electrode segments 28a and/or 28b substantially within anterior mediastinum 36, may depend upon a number of factors, including location, type, size, orientation, and/or spacing of electrodes 32a and 32b and/or electrode segments 28a and 28b, location of ICD 9 relative to electrodes 32a and 32b and/or electrode segments 28a and 28b, physical abnormalities of the heart (e.g., pericardial adhesions or myocardial infarctions), or other factor(s).

Processing circuitry 280 may include a timing and control circuitry, which may be embodied as hardware, firmware, software, or any combination thereof. The timing and control circuitry may comprise a dedicated hardware circuit, such as an ASIC, separate from other processing circuitry 280 components, such as a microprocessor, or a software module executed by a component of processing circuitry 280, which may be a microprocessor or ASIC. The timing and control circuitry may implement programmable counters. If ICD 9 is configured to generate and deliver pacing pulses to heart 26, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control circuitry within processing circuitry 280 may include atrial and ventricular pacing escape intervals, refractory periods during which sensed P-waves and R-waves are ineffective to restart timing of the escape intervals, and the pulse widths of the pacing pulses. As another example, the timing and control circuitry may withhold sensing from one or more channels of sensing circuitry 286 for a time interval during and after delivery of electrical stimulation to heart 26. The durations of these intervals may be determined by processing circuitry 264 in response to stored data in memory 282. The timing and control circuitry of processing circuitry 264 may also determine the amplitude of the cardiac pacing pulses.

Interval counters implemented by the timing and control circuitry of processing circuitry 280 may be reset upon sensing of R-waves and P-waves with detection channels of sensing circuitry 286. In examples in which ICD 9 provides pacing, therapy delivery circuitry 288 may include pacer output circuits that are coupled to electrodes, for example, appropriate for delivery of a bipolar or unipolar pacing pulse to one of the chambers of heart 26. In such examples, processing circuitry 280 may reset the interval counters upon the generation of pacing pulses by therapy delivery circuitry 288, and thereby control the basic timing of cardiac pacing functions, including ATP or post-shock pacing.

The value of the count present in the interval counters when reset by sensed R-waves and P-waves may be used by processing circuitry 280 to measure the durations of R-R intervals, P-P intervals, P-R intervals and R-P intervals, which are measurements that may be stored in memory 282. Processing circuitry 280 may use the count in the interval counters to detect a tachyarrhythmia event, such as atrial fibrillation, atrial tachycardia, ventricular fibrillation, or ventricular tachycardia. These intervals may also be used to detect the overall heart rate, ventricular contraction rate, and heart rate variability. A portion of memory 282 may be configured as a plurality of recirculating buffers, capable of holding series of measured intervals, which may be analyzed by processing circuitry 280 in response to the occurrence of a pace or sense interrupt to determine whether the patient's heart 26 is presently exhibiting atrial or ventricular tachyarrhythmia.

In some examples, an arrhythmia detection method may include any suitable tachyarrhythmia detection algorithms. In some examples, processing circuitry 280 may determine that tachyarrhythmia has occurred by identification of shortened R-R (or P-P) interval lengths. Generally, processing circuitry 280 detects tachycardia when the interval length falls below 220 milliseconds and fibrillation when the interval length falls below 180 milliseconds. In other examples, processing circuitry 280 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 282. This interval length may need to be detected for a certain number of consecutive cycles, for a certain percentage of cycles within a running window, or a running average for a certain number of cardiac cycles, as examples. In other examples, additional physiological parameters may be used to detect an arrhythmia. For example, processing circuitry 280 may analyze one or more morphology measurements, impedances, or any other physiological measurements to determine that patient 14 is experiencing a tachyarrhythmia.

In the event that an ATP regimen is desired, timing intervals for controlling the generation of ATP therapies by therapy deliver circuitry 288 may be loaded by processing circuitry 280 into the timing and control circuitry based on ATP parameters 276 to control the operation of the escape interval counters therein and to define refractory periods during which detection of R-waves and P-waves is ineffective to restart the escape interval counters for the ATP. An ATP regimen may be desired if processing circuitry 280 detects an atrial or ventricular tachyarrhythmia based on signals from sensing circuitry 286, and/or receives a command from another device or system, such as IPD 16, as examples.

Memory 282 may be configured to store a variety of operational parameters, therapy parameters, including ATP therapy parameters, sensed and detected data, and any other information related to the therapy and treatment of patient 14. In the example of FIG. 6, memory 282 may store sensed ECGs, detected arrhythmias, communications from IPD 16, and therapy parameters that define ATP therapy (which may be stored in parameters/rules criteria 294). In other examples, memory 282 may act as a temporary buffer for storing data until it can be uploaded to IPD 16, another implanted device, or external device 21.

Communication circuitry 284 includes any suitable hardware, firmware, software or any combination thereof for communicating with another device, such as external device 21 (FIGS. 1A-1C and 7), IPD 16 (FIGS. 1A-1C and FIG. 2), a clinician programmer, a patient monitoring device, or the like. For example, communication circuitry 284 may include appropriate modulation, demodulation, frequency conversion, filtering, and amplifier components for transmission and reception of data with the aid of antenna 292. Antenna 292 may be located within connector block of ICD 9 or within the housing of ICD 9. Under the control of processing circuitry 280, communication circuitry 284 may receive downlink telemetry from and send uplink telemetry to external device 21 with the aid of antenna 292, which may be internal and/or external. Processing circuitry 280 may provide the data to be uplinked to external device 21 and the control signals for the telemetry circuit within communication circuitry 284, e.g., via an address/data bus. In some examples, communication circuitry 284 may provide received data to processing circuitry 280 via a multiplexer.

In some examples, ICD 9 may signal external device 21 to further communicate with and pass the alert through a network such as the Medtronic CareLink® Network developed by Medtronic plc, of Dublin, Ireland, or some other network linking patient 14 to a clinician. ICD 9 may spontaneously transmit information to the network or in response to an interrogation request from a user.

Power source 290 may be any type of device that is configured to hold a charge to operate the circuitry of ICD 9. Power source 290 may be provided as a rechargeable or non-rechargeable battery. In other example, power source 290 may incorporate an energy scavenging system that stores electrical energy from movement of ICD 9 within patient 14.

The various circuitry of ICD 9 may include any one or more processors, controllers, DSPs, ASICs, FPGAs, or equivalent discrete or integrated circuitry, including analog circuitry, digital circuitry, or logic circuitry.

FIG. 7 is a functional block diagram illustrating an example configuration of external device 21 of FIGS. 1A-1C and FIG. 3. External device 21 may include processing circuitry 400, memory 402, communication circuitry 408, user interface 406, motion sensor(s) 410 (which may include one or more accelerometers), and power source 404. Processing circuitry 400 controls user interface 406 and communication circuitry 408, and stores and retrieves information and instructions to and from memory 402. External device 21 may be configured for use as a clinician programmer or a patient programmer. Processing circuitry 400 may comprise any combination of one or more processors including one or more microprocessors, DSPs, ASICs, FPGAs, or other equivalent integrated or discrete logic circuitry. Accordingly, processing circuitry 400 may include any suitable structure, whether in hardware, software, firmware, or any combination thereof, to perform the functions ascribed herein to processing circuitry 400.

In some examples, motion sensor(s) 410 may include a three-axis accelerometer, one or more single-axis accelerometers, or the like. In examples, where external device 21 may be a smart phone carried by patient 14 (e.g., in a pocket of patient 14), a wearable device, or the like, motion sensor(s) 410 may be configured to generate one or more signals that may be indicative of a posture of patient 14, for example, an upright posture, a slumping posture, a non-upright posture, or the like. Additionally, or alternatively, motion sensor(s) 298 may be configured to generate a spike in one or more signals that may be indicative of a fall. For example, the spike may correspond to an acceleration of patient 14 towards the ground when falling, or a spike may correspond to an impact with the ground caused by falling.

Processing circuitry 400 may monitor the one or more signals of motion sensor(s) 410 to determine whether patient 14 has fallen, slumping, or otherwise changed from an upright posture to a non-upright posture. In some examples, processing circuitry 400 may affect the modulation of at least one of a therapy delivery or an indication, such as an alert, in response to a determination that patient 14 has fallen, slumping, or otherwise changed from an upright posture to a non-upright posture. For example, processing circuitry 400 may interact with one or more IMDs via communication circuitry 408 to receive an indication of a cardiac event and, in response to determining that patient 14 has fallen, slumping, or otherwise changed from an upright posture to a non-upright posture, control the one or more IMDs to modulate the delivery of therapy to patient 14, and/or deliver an alert regarding patient 14.

In some examples, upon determining that patient 14 has fallen, slumping, or otherwise changed from an upright posture to a non-upright posture, processing circuitry 400 may relax or override rules of a cardiac rhythm discrimination algorithm and control therapy delivery circuitry to deliver a shock or other therapy to heart 26 of patient 14. Such rules may be stored in parameters/rules/criteria 412 of memory 402. In some examples, upon determining that patient 14 has fallen, slumping, or otherwise changed from an upright posture to a non-upright posture, processing circuitry 400 may relax onset detection criteria, which may be stored in parameters/rules/criteria 412, to make it more likely that processing circuitry 280 may detect a cardiac event. In some examples, upon relaxing or overriding the rules or relaxing the onset detection criteria, processing circuitry 400 may control communication circuitry 408 to communicate with an IMD to transmit information regarding the relaxing or overriding the rules or relaxing the onset detection criteria. In some examples, upon relaxing or overriding the rules or relaxing the onset detection criteria, processing circuitry 400 determine that therapy should be delivered, based on the relaxed or overridden rules or the relaxed onset detection criteria, may control communication circuitry 408 to transmit a message to an IMD to deliver therapy.

A user, such as a clinician or patient 14, may interact with external device 21 through user interface 406. User interface 406 may include a display, such as an LCD or LED display or other type of screen, to present information related the ATP therapy, including ATP therapy parameters stored in parameters/rules/criteria 412 or in memory of an IMD, or to display a visual indication or provide audio indication relating to a cardiac event to the user. In addition, user interface 406 may include an input mechanism to receive input from the user. The input mechanisms may include, for example, buttons, a keypad (e.g., an alphanumeric keypad), a peripheral pointing device or another input mechanism that allows the user to navigate through user interfaces presented by processing circuitry 400 of external device 21 and provide input.

If external device 21 includes buttons and a keypad, the buttons may be dedicated to performing a certain function, i.e., a power button, or the buttons and the keypad may be soft keys that change in function depending upon the section of the user interface currently viewed by the user. Alternatively, a screen of external device 21 may be a touch screen that allows the user to provide input directly to the user interface shown on the display. The user may use a stylus or a finger to provide input to the display. In other examples, user interface 406 also includes audio circuitry for providing audible instructions or sounds, such as an indication regarding a cardiac event, to patient 14 and/or receiving voice commands from patient 14, which may be useful if patient 14 has limited motor functions. Patient 14, a clinician or another user may also interact with external device 21 to manually select therapy programs, generate new therapy programs, modify therapy programs through individual or global adjustments, and transmit the new programs to PCD 110, IPD 16, and/or ICD 9.

In some examples, at least some of the control of therapy delivery by PCD 110, ICD 9, and/or IPD 16 may be implemented by processing circuitry 400 of external device 21. For example, in some examples, processing circuitry 400 may control delivery of cardiac therapy, such as ATP therapy by PCD 110, ICD 9, and/or IPD 16 by communicating with by PCD 110, ICD 9, and/or IPD 16 and may receive data regarding sensed signals and by communicating with by PCD 110, ICM 300, ICD 9, and/or IPD 16 to control therapy delivery circuitry of any of by PCD 110, ICD 9, and/or IPD 16. In some examples, memory 402 may store ATP therapy parameters, may use the parameters to control therapy delivery circuitry of by PCD 110, ICD 9, and/or IPD 16, and/or may modify ATP therapy parameters.

Memory 402 may include instructions for operating user interface 406 and communication circuitry 408, for managing power source 404, and for performing any functions attributed to external device 21 (e.g., determining a cardiac event, determining a fall, a slumping posture, or a change from an upright posture to a non-upright posture, controlling the delivery of therapy, and/or generating an indication for output). Memory 402 may also store any therapy data retrieved from PCD 110 during the course of therapy, accelerometer data, or other sensed data. The clinician may use this therapy data to determine the progression of the patient condition in order to predict future treatment. Memory 402 may include any volatile or nonvolatile memory, such as RAM, ROM, FRAM, EEPROM or flash memory. Memory 402 may also include a removable memory portion that may be used to provide memory updates or increases in memory capacities. A removable memory may also allow sensitive patient data to be removed before external device 21 is used by a different patient.

Wireless telemetry in external device 21 may be accomplished by RF communication or proximal inductive interaction of external device 21 with PCD 110, ICM 300, ICD 9, and/or IPD 16. This wireless communication is possible through the use of communication circuitry 408, which may communicate with a proprietary protocol or industry-standard protocol such as using the Bluetooth specification set. Accordingly, communication circuitry 408 may be similar to the communication circuitry contained within by PCD 110, ICD 9, and/or IPD 16. In alternative examples, external device 21 may be capable of infrared communication or direct communication through a wired connection. In this manner, other external devices may be capable of communicating with external device 21 without needing to establish a secure wireless connection.

Power source 404 may deliver operating power to the components of external device 21. Power source 404 may include a battery and a power generation circuit to produce the operating power. In some examples, the battery may be rechargeable to allow extended operation. Recharging may be accomplished by electrically coupling power source 404 to a cradle or plug that is connected to an AC outlet. In addition, recharging may be accomplished through proximal inductive interaction between an external charger and an inductive charging coil within external device 21. In other examples, traditional batteries (e.g., nickel cadmium or lithium ion batteries) may be used. In addition, external device 21 may be directly coupled to an alternating current outlet to operate. Power source 404 may include circuitry to monitor power remaining within a battery. In this manner, user interface 406 may provide a current battery level indicator or low battery level indicator when the battery needs to be replaced or recharged. In some cases, power source 404 may be capable of estimating the remaining time of operation using the current battery.

According to the techniques of this disclosure, processing circuitry 400 may determine the occurrence of the cardiac event in patient 14. Processing circuitry 400 may determine, based at least in part on at least one accelerometer signal, at least one of a fall of patient 14, a slumping posture of patient 14, or a change from an upright posture to a non-upright posture of patient 14. Processing circuitry 400 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, processing circuitry 400 may modulate a response to the cardiac event.

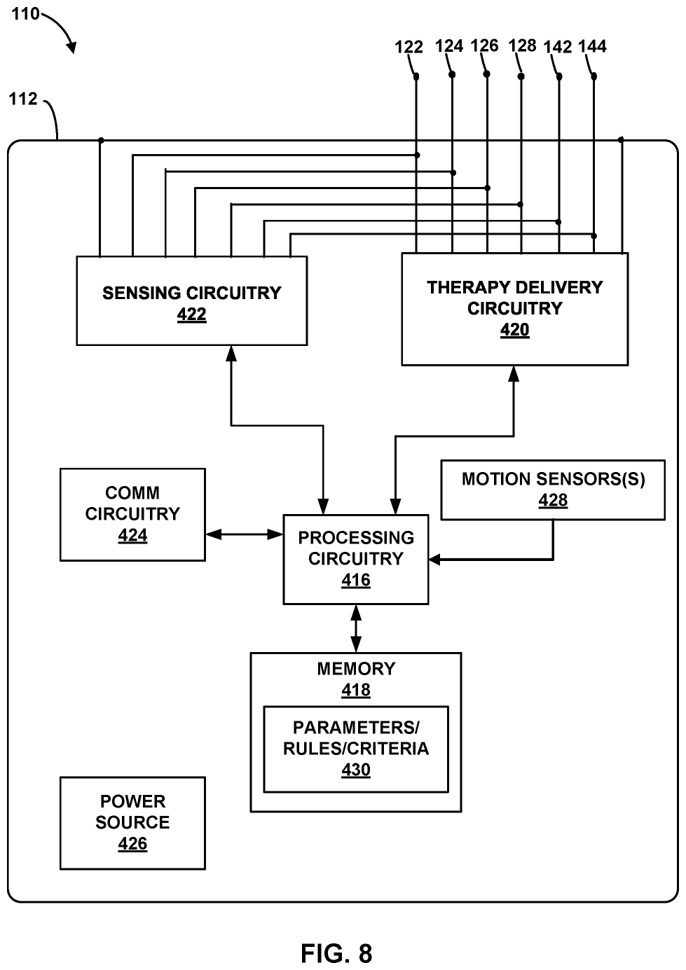

FIG. 8 is a functional block diagram illustrating an example configuration of pacemaker/cardioverter/defibrillator (PCD) 110 of the implantable medical device system of FIG. 3. As illustrated in FIG. 8, in one example, PCD 110 includes sensing circuitry 422, therapy delivery circuitry 420, processing circuitry 416 and associated memory 418, communication circuitry 424, motion sensor(s) 428 (which may include one or more accelerometers), and power source 426. The electronic components may receive power from power source 426, which may be a rechargeable or non-rechargeable battery. In other examples, PCD 110 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components.

Sensing circuitry 422 receives cardiac electrical signals from electrodes 112, 122, 124, 126, 128, 130, 132, 136, 142 and 144 carried by the right ventricular lead 118, left ventricular lead 120 and/or atrial lead 121, along with a housing electrode 112 associated with the housing 113, for sensing cardiac events attendant to the depolarization of myocardial tissue, e.g., P-waves and R-waves. Sensing circuitry 422 may also be configured to sense fiducial points and QRS complexes. Sensing circuitry 422 may include a switch circuitry for selectively coupling electrodes 122, 124, 126, 128, 142, 144, and housing electrode 112 to sensing circuitry 422 in order to monitor electrical activity of heart 116. The switch circuitry may include a switch array, switch matrix, multiplexer, or any other type of switching device suitable to selectively couple one or more of the electrodes to sensing circuitry 422. In some examples, processing circuitry 416 selects the electrodes to function as sense electrodes, or the sensing vector, via the switch circuitry within sensing circuitry 422.

Sensing circuitry 422 may include multiple sensing channels, each of which may be selectively coupled to respective combinations of electrodes 122, 124, 126, 128, 142, 144 and housing 113 to detect electrical activity of a particular chamber of heart 116, e.g., an atrial sensing channel and a ventricular sensing channel. Each sensing channel may comprise a sense amplifier that outputs an indication to processing circuitry 416 in response to sensing of a cardiac depolarization, in the respective chamber of heart 116. In this manner, processing circuitry 416 may receive sense event signals corresponding to the occurrence of sensed R-waves and P-waves in the respective chambers of heart 116. Sensing circuitry 422 may further include digital signal processing circuitry for providing processing circuitry 416 with digitized ECG signals, which may be used for cardiac rhythm discrimination.

The components of sensing circuitry 422 may be analog components, digital components or a combination thereof. Sensing circuitry 422 may, for example, include one or more sense amplifiers, filters, rectifiers, threshold detectors, ADCs or the like. Sensing circuitry 422 may convert the sensed signals to digital form and provide the digital signals to processing circuitry 416 for processing or analysis. For example, sensing circuitry 422 may amplify signals from the sensing electrodes and convert the amplified signals to multi-bit digital signals by an ADC. Sensing circuitry 422 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 416.

Sensing circuitry 422 and/or processing circuitry 416 may also include circuitry for measuring the capture threshold for the delivery of pacing pulses via electrodes 122, 124, 126, 128, 142, 144, and 112. The capture threshold may indicate the voltage and pulse width necessary to induce depolarization of the surrounding cardiac muscle. For example, processing circuitry 416 may periodically control therapy delivery circuitry 420 to modify the amplitude of pacing pulses delivered to a patient, and sensing circuitry 422 and/or processing circuitry 416 may detect whether the surrounding cardiac tissue depolarized in response to the pacing pulses, i.e., detected whether there was an evoked response to the pacing pulse. Processing circuitry 416 may determine the capture threshold based on the amplitude where loss of capture occurred. In addition to detecting and identifying specific types of cardiac rhythms, sensing circuitry 422 may also sample the detected intrinsic signals to generate an ECG or other time-based indication of cardiac events.

Processing circuitry 416 may process the signals from sensing circuitry 422 to monitor electrical activity of the heart of the patient. Processing circuitry 416 may store signals obtained by sensing circuitry 422 as well as any generated ECG waveforms, marker channel data, accelerometer data, or other data derived based on the sensed signals in memory 418. Processing circuitry 416 may analyze the ECG waveforms and/or marker channel data to detect cardiac events (e.g., tachycardia), determine whether a fall, a slumping posture, or a change from an upright posture to a non-upright posture occurred in patient 14 that is associated with the cardiac event. In response to determining that the fall, the slumping posture, or the change from an upright posture to a non-upright posture is associated with the cardiac event, processing circuitry 416 may modulate control therapy delivery circuitry 420 to modulate delivery of the desired therapy to treat the cardiac event, e.g., ATP therapy, and/or control communication circuitry 424 to modulate the generation and/or delivery of an indication relating to the cardiac event.

In examples in which PCD 110 includes more than two electrodes, therapy delivery circuitry 420 may include a switch and processing circuitry 416 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes. Processing circuitry 416 may select the electrodes to function as signal electrodes, or the signal vector, via the switch circuitry within therapy delivery circuitry 420. In some instances, the same switch circuitry may be used by both therapy delivery circuitry 420 and sensing circuitry 422. In other instances, each of sensing circuitry 422 and therapy delivery circuitry 420 may have separate switch circuitry.

Memory 418 may include computer-readable instructions that, when executed by processing circuitry 416, cause PCD 110 to perform various functions attributed throughout this disclosure to PCD 110 and processing circuitry 416 (e.g., determining a cardiac event, determining a fall, a slumping posture, or a change from an upright posture to a non-upright posture, delivering therapy, and/or generating an indication for output). The computer-readable instructions may be encoded within memory 418. Memory 418 may comprise computer-readable storage media including any volatile, non-volatile, magnetic, optical, or electrical media, such as a RAM, ROM, FRAM, NVRAM, EEPROM, flash memory, or any other digital media.

Processing circuitry 416 may include any one or more of a microprocessor, a controller, a DSP, an ASIC, an FPGA, or equivalent discrete or integrated logic circuitry or state machine. In some examples, processing circuitry 416 may include multiple components, such as any combination of one or more microprocessors, one or more controllers, one or more DSPs, one or more ASICs, or one or more FPGAs, as well as other discrete or integrated logic circuitry or state machines. The functions attributed to processing circuitry 416 herein may be embodied as software, firmware, hardware or any combination thereof.

Therapy delivery circuitry 420 is electrically coupled to electrodes 122, 124, 126, 128, 142, 144, and 112. In the illustrated example, therapy delivery circuitry 420 is configured to generate and deliver electrical stimulation therapy to a heart of a patient. For example, therapy delivery circuitry 420 may deliver the electrical stimulation therapy to a portion of cardiac muscle within the heart via any combination of electrodes 122, 124, 126, 128, 142, 144, and 112. In some examples, therapy delivery circuitry 420 may deliver pacing stimulation, e.g., ATP therapy, in the form of voltage or current electrical pulses. In other examples, therapy delivery circuitry 420 may deliver stimulation in the form of other signals, such as sine waves, square waves, or other substantially continuous time signals.

Although PCD 110 is generally described as delivering pacing pulses, PCD 110 may deliver cardioversion or defibrillation pulses in other examples. Therapy delivery circuitry 420 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 420 may include a first set of components configured to provide pacing therapy and a second set of components configured to provide defibrillation therapy. In other instances, therapy delivery circuitry 420 may utilize the same set of components to provide both pacing and defibrillation therapy. In still other instances, therapy delivery circuitry 420 may share some of the defibrillation and pacing therapy components while using other components solely for defibrillation or pacing.

Motion sensor(s) 428 may include a three-axis accelerometer, one or more single-axis accelerometers, or the like. Motion sensor(s) 428 may be configured to generate one or more signals that may be indicative of a posture of patient 14, for example, an upright posture, a slumping posture, a non-upright posture, or the like. Additionally, or alternatively, motion sensor(s) 428 may be configured to generate a spike in one or more signals that may be indicative of a fall. For example, the spike may correspond to an acceleration of patient 14 towards the ground when falling, or a spike may correspond to an impact with the ground caused by falling.

Processing circuitry 416 may monitor the one or more signals of motion sensor(s) 428 to determine whether patient 14 has fallen, slumping, or otherwise changed from an upright posture to a non-upright posture. In some examples, processing circuitry 416 may modulate at least one of a therapy delivery or an alert in response to a determination that patient 14 has fallen, slumping, or otherwise changed from an upright posture to a non-upright posture.

In some examples, upon determining that patient 14 has fallen, slumping, or otherwise changed from an upright posture to a non-upright posture, processing circuitry 416 may relax or override rules of a cardiac rhythm discrimination algorithm and control therapy delivery circuitry to deliver a shock or other therapy to heart 26 of patient 14. Such rules may be stored in parameters/rules/criteria 430 of memory 418. In some examples, upon determining that patient 14 has fallen, slumping, or otherwise changed from an upright posture to a non-upright posture, processing circuitry 416 may relax onset detection criteria, which may be stored in parameters/rules/criteria 430, to make it more likely that processing circuitry 416 may detect and treat a cardiac event.

Processing circuitry 416 may control therapy delivery circuitry 420 to deliver electrical stimulation therapy, e.g., anti-tachyarrhythmia therapy, post-shock pacing, etc., to heart 116 according to therapy parameters, which may be stored in parameters/rules/criteria 430 in memory 418. Therapy delivery circuitry 420 is electrically coupled to electrodes 122, 124, 126, 128, 142, 144 and housing electrode 112 (all of which are shown in FIGS. 3 and 8). Therapy delivery circuitry 420 is configured to generate and deliver electrical stimulation therapy to heart 116 via selected combinations of electrodes 122, 124, 126, 128, 142, 144, and housing electrode 112.

Processing circuitry 416 may control therapy delivery circuitry 420 to deliver pacing pulses for ATP therapy according to ATP parameters stored in parameters/rules/criteria 430. ATP therapy parameters may include pulse intervals, pulse width, current and/or voltage amplitudes, and durations for each pacing mode. In some examples, ATP parameters may include a plurality of ranges of propagation times and associated number of pulses of an ATP train, a formula that may be applied to a first propagation time, a linear equation that may be applied to a first propagation time, a continuous function that may be applied to a first propagation time, or a look-up table that may include first propagation times and associated numbers of pulses of an ATP train. For example, the pulse interval may be based on a fraction of the detected ventricular tachycardia cycle length and be between approximately 150 milliseconds and 500 milliseconds (e.g., between approximately 2.0 hertz and 7.0 hertz), and the pulse width may be between approximately 0.5 milliseconds and 2.0 milliseconds. The amplitude of each pacing pulse may be between approximately 2.0 volts and 10.0 volts. In some examples, the pulse amplitude may be approximately 6.0 V and the pulse width may be approximately 1.5 milliseconds; another example may include pulse amplitudes of approximately 5.0 V and pulse widths of approximately 1.0 milliseconds.

Each pulse, or burst of pulses, may include a ramp up in amplitude or in pulse rate. In addition, trains of pulses in successive ATP periods may be delivered at increasing pulse rate in an attempt to capture the heart and terminate the tachycardia.

Processing circuitry 416 controls therapy delivery circuitry 420 to generate and deliver pacing pulses with any of a number of shapes, amplitudes, pulse widths, or other characteristic to capture the heart. For example, the pacing pulses may be monophasic, biphasic, or multi-phasic (e.g., more than two phases). The pacing thresholds of the heart when delivering pacing pulses may depend upon a number of factors, including location, type, size, orientation, and/or spacing of PCD 110 and/or electrodes 122, 124, 126, 128, 142, 144, and 122, physical abnormalities of the heart (e.g., pericardial adhesions or myocardial infarctions), or other factor(s).

In examples in which PCD 110 includes more than two electrodes, therapy delivery circuitry 420 may include a switch and processing circuitry 416 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes.

Memory 418 stores parameters/rules/criteria 430, including intervals, counters, or other data used by processing circuitry 416 to control the delivery of pacing pulses by therapy delivery circuitry 420. Such data may include intervals and counters used by processing circuitry 416 to control the delivery of pacing pulses to heart 116. The intervals and/or counters are, in some examples, used by processing circuitry 416 to control the timing of delivery of pacing pulses relative to an intrinsic or paced event in another chamber. ATP therapy parameters may also include intervals for controlling cardiac sensing functions such as blanking intervals and refractory sensing intervals and counters for counting sensed events for detecting cardiac rhythm episodes. Events sensed by sense amplifiers included in sensing circuitry 422 are identified in part based on their occurrence outside a blanking interval and inside or outside of a refractory sensing interval. Events that occur within predetermined interval ranges are counted for detecting cardiac rhythms. According to examples described herein, sensing circuitry 422, memory 418, and processing circuitry 416 are configured to use timers and counters for measuring sensed event intervals and determining event patterns for use in detecting possible ventricular lead dislodgement.

Memory 418 may be further configured to store sensed and detected data, and any other information related to the therapy and treatment of a patient. In the example of FIG. 8, memory 418 may store sensed ECGs, detected arrhythmias, accelerometer signals, and communications from ICM 300 and/or external device 21. In other examples, memory 418 may act as a temporary buffer for storing data until it can be uploaded to another implanted device, or external device 21.

Communication circuitry 424 is used to communicate with external device 21 and/or ICM 300 for transmitting data accumulated by PCD 110 and for receiving interrogation and programming commands to and/or from external device 21 and/or ICM 300. Communication circuitry 268 includes any suitable hardware, firmware, software or any combination thereof for communicating with another device, such as external device 21 (FIGS. 1A-1C and 7), ICM 300 (FIGS. 3 and 9), a clinician programmer, a patient monitoring device, or the like. For example, communication circuitry 424 may include appropriate modulation, demodulation, frequency conversion, filtering, and amplifier components for transmission and reception of data. Under the control of processing circuitry 416, communication circuitry 424 may receive downlink telemetry from and send uplink telemetry to external device 21 with the aid of an antenna, which may be internal and/or external. Processing circuitry 416 may provide the data to be uplinked to external device 21 and the control signals for the telemetry circuit within communication circuitry 424, e.g., via an address/data bus. In some examples, communication circuitry 424 may provide received data to processing circuitry 416 via a multiplexer.

In some examples, PCD 110 may signal external device 21 to further communicate with and pass the alert through a network such as the Medtronic CareLink® Network developed by Medtronic plc, of Dublin, Ireland, or some other network linking a patient to a clinician. PCD 110 may spontaneously transmit information to the network or in response to an interrogation request from a user.

Power source 426 may be any type of device that is configured to hold a charge to operate the circuitry of PCD 110. Power source 426 may be provided as a rechargeable or non-rechargeable battery. In other example, power source 426 may incorporate an energy scavenging system that stores electrical energy from movement of PCD 110 within patient 114.

The various circuitry of PCD 110 may include any one or more processors, controllers, DSPs, ASICs, FPGAs, or equivalent discrete or integrated circuitry, including analog circuitry, digital circuitry, or logic circuitry.

According to the techniques of this disclosure, processing circuitry 416 may determine the occurrence of the cardiac event in patient 14. Processing circuitry 416 may determine, based at least in part on at least one accelerometer signal, at least one of a fall of patient 14, a slumping posture of patient 14, or a change from an upright posture to a non-upright posture of patient 14. Processing circuitry 416 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, processing circuitry 416 may modulate a response to the cardiac event.

FIG. 9 is a functional block diagram illustrating an example configuration of ICM 300 of FIGS. 3-4. In the illustrated example, ICM 300 includes processing circuitry 500, memory 502, sensing circuitry 506, communication circuitry 508 connected to antenna 322, motion sensor(s) 512 (which may include one or more accelerometers), and power source 510. The electronic components may receive power from a power source 510, which may be a rechargeable or non-rechargeable battery. In other examples, ICM 300 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components.

Memory 502 includes computer-readable instructions that, when executed by processing circuitry 500, cause ICM 300 and processing circuitry 500 to perform various functions attributed to ICM 300 and processing circuitry 500 herein (e.g., determining a cardiac event, determining a fall, a slumping posture, or a change from an upright posture to a non-upright posture, and/or generating an indication for output). Memory 502 may include any volatile, non-volatile, magnetic, optical, or electrical media, such as RAM, ROM, FRAM, NVRAM, EEPROM, flash memory, or any other digital or analog media.

Processing circuitry 500 may include any one or more of a microprocessor, a controller, a DSP, an ASIC, an FPGA, or equivalent discrete or analog logic circuitry. In some examples, processing circuitry 500 may include multiple components, such as any combination of one or more microprocessors, one or more controllers, one or more DSPs, one or more ASICs, or one or more FPGAs, as well as other discrete or integrated logic circuitry. The functions attributed to processing circuitry 500 herein may be embodied as software, firmware, hardware or any combination thereof.

Motion sensor(s) 512 may include a three-axis accelerometer, one or more single-axis accelerometers, or the like. Motion sensor(s) 512 may be configured to generate one or more signals that may be indicative of a posture of patient 14, for example, an upright posture, a slumping posture, a non-upright posture, or the like. Additionally, or alternatively, motion sensor(s) 512 may be configured to generate a spike in one or more signals that may be indicative of a fall. For example, the spike may correspond to an acceleration of patient 14 towards the ground when falling, or a spike may correspond to an impact with the ground caused by falling.

Processing circuitry 500 may monitor the one or more signals of motion sensor(s) 512 to determine whether patient 14 has fallen, slumping, or otherwise changed from an upright posture to a non-upright posture. In some examples, processing circuitry 500 may modulate at least one of a therapy delivery or an alert in response to a determination that patient 14 has fallen, slumping, or otherwise changed from an upright posture to a non-upright posture. For example, processing circuitry 500 may modulate the generation of an indication of a cardiac event. In some examples, processing circuitry 500 may interact with one or more other IMDs via communication circuitry 508 to transmit an indication that patient 14 has fallen, is in a slumping posture, or otherwise changed from an upright posture to a non-upright posture, and the one or more other IMDs may modulate delivery of therapy or the generation of an indication of a cardiac event based on the indication that patient 14 has fallen, is in a slumping posture, or otherwise changed from an upright posture to a non-upright posture.

In some examples, upon determining that patient 14 has fallen, slumping, or otherwise changed from an upright posture to a non-upright posture, processing circuitry 500 may relax or override rules of a cardiac rhythm discrimination algorithm. Such rules may be stored in parameters/rules/criteria 514 of memory 502. In some examples, upon determining that patient 14 has fallen, slumping, or otherwise changed from an upright posture to a non-upright posture, processing circuitry 500 may relax onset detection criteria, which may be stored in parameters/rules/criteria 514, to make it more likely that processing circuitry 500 may detect a cardiac event. In some examples, upon relaxing or overriding the rules or relaxing the onset detection criteria, processing circuitry 500 may control communication circuitry 508 to communicate with an IMD to transmit information regarding the relaxing or overriding the rules or relaxing the onset detection criteria. In some examples, upon relaxing or overriding the rules or relaxing the onset detection criteria, processing circuitry 500 determine that therapy should be delivered, based on the relaxed or overridden rules or the relaxed onset detection criteria and may control communication circuitry 508 to transmit a message to an IMD to deliver therapy.

Processing circuitry 500 controls sensing circuitry 506 to sense evoked responses of the heart via electrodes 304 and 306. Although ICM 300 may only include two electrodes, e.g., electrodes 304 and 306, ICM 300 may utilize three or more electrodes in other examples. ICM 300 may use any combination of electrodes to detect electrical signals from patient 114. Sensing circuitry 506 is electrically coupled to electrodes 304 and 306 carried on housing 302 of ICM 300.

Sensing circuitry 506 is electrically connected to and monitors signals from one or more of electrodes 304 and 306 in order to monitor electrical activity of a heart, impedance, or other electrical phenomenon. Sensing may be done to determine heart rates or heart rate variability, or to detect arrhythmias (e.g., tachyarrhythmias or bradycardia) or other electrical signals. Sensing circuitry 504 may also include a switch to select which of the available electrodes (or electrode polarity) are used to sense the heart activity, depending upon which electrode combination, or electrode vector, is used in the current sensing configuration. In examples with several electrodes, processing circuitry 500 may select the electrodes that function as sense electrodes, i.e., select the sensing configuration, via the switch circuitry within sensing circuitry 504. Sensing circuitry 504 may include one or more detection channels, each of which may be coupled to a selected electrode configuration for detection of cardiac signals via that electrode configuration. Some detection channels may be configured to detect cardiac events, such as fiducial points, QRS complexes, P- or R-waves, and provide indications of the occurrences of such events to processing circuitry 500 Processing circuitry 500 may control the functionality of sensing circuitry 506 by providing signals via a data/address bus.

The components of sensing circuitry 506 may be analog components, digital components or a combination thereof. Sensing circuitry 506 may, for example, include one or more sense amplifiers, filters, rectifiers, threshold detectors, ADCs, or the like. Sensing circuitry 506 may convert the sensed signals to digital form and provide the digital signals to processing circuitry 500 for processing or analysis. For example, sensing circuitry 506 may amplify signals from the sensing electrodes and convert the amplified signals to multi-bit digital signals by an ADC. Sensing circuitry 506 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 500.

Processing circuitry 500 may implement programmable counters and may withhold sensing from one or more channels of sensing circuitry 506 for a time interval during and after delivery of electrical stimulation to heart 116. The durations of these intervals may be determined by processing circuitry 500 in response to stored data in memory 502.

Interval counters implemented by processing circuitry 500 may be reset upon sensing of R-waves and P-waves with detection channels of sensing circuitry 506. The value of the count present in the interval counters when reset by sensed R-waves and P-waves may be used by processing circuitry 500 to measure the durations of R-R intervals, P-P intervals, P-R intervals and R-P intervals, which are measurements that may be stored in memory 502. Processing circuitry 500 may use the count in the interval counters to detect a tachyarrhythmia event, such as atrial fibrillation, atrial tachycardia, ventricular fibrillation, or ventricular tachycardia. These intervals may also be used to detect the overall heart rate, ventricular contraction rate, and heart rate variability. A portion of memory 502 may be configured as a plurality of recirculating buffers, capable of holding series of measured intervals, which may be analyzed by processing circuitry 500 in response to the occurrence of a sense interrupt to determine whether the patient's heart 116 is presently exhibiting atrial or ventricular tachyarrhythmia.

In some examples, an arrhythmia detection method may include any suitable tachyarrhythmia detection algorithms. In some examples, methodologies that utilize timing and morphology of the ECG, may be employed by processing circuitry 500 in other examples.

In some examples, processing circuitry 500 may determine that tachyarrhythmia has occurred by identification of shortened R-R (or P-P) interval lengths. Generally, processing circuitry 500 detects tachycardia when the interval length falls below 220 milliseconds and fibrillation when the interval length falls below 180 milliseconds. In other examples, processing circuitry 500 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 502. This interval length may need to be detected for a certain number of consecutive cycles, for a certain percentage of cycles within a running window, or a running average for a certain number of cardiac cycles, as examples. In other examples, additional physiological parameters may be used to detect an arrhythmia. For example, processing circuitry 500 may analyze one or more morphology measurements, impedances, or any other physiological measurements to determine that patient 114 is experiencing a tachyarrhythmia.

In addition to detecting and identifying specific types of cardiac rhythms, sensing circuitry 504 may also sample the detected intrinsic signals to generate an ECG or other time-based indication of cardiac events. Processing circuitry 500 may also be able to coordinate the delivery of pacing pulses from another device, such as PCD 110. For example, processing circuitry 500 may identify delivered pulses from PCD 110 via sensing circuitry 506 and update pulse timing to accomplish a selected pacing regimen. This detection may be on a pulse-to-pulse or beat-to-beat basis, or on a less frequent basis to make slight modifications to pulse rate over time. In other examples, IPDs may communicate with each other via communication circuitry 508 and/or instructions over a carrier wave (such as a stimulation waveform). In this manner, ATP pacing may be coordinated by multiple devices.

Memory 502 may be configured to store a variety of sensed and detected data and any other information related to the therapy and treatment of patient 114. In the example of FIG. 9, memory 502 may store sensed ECGs and/or detected arrhythmias, accelerometer signals, and/or communications from PCD 110 and/or external device 21. In other examples, memory 502 may act as a temporary buffer for storing data until it can be uploaded to PCD 110, another implanted device, or external device 21.

Communication circuitry 508 includes any suitable hardware, firmware, software or any combination thereof for communicating with another device, such as external device 21, PCD 110 (FIGS. 1A-1C), a clinician programmer, a patient monitoring device, or the like. For example, communication circuitry 508 may include appropriate modulation, demodulation, frequency conversion, filtering, and amplifier components for transmission and reception of data. Under the control of processing circuitry 500, communication circuitry 508 may receive downlink telemetry from and send uplink telemetry to external device 21 with the aid of antenna 322, which may be internal and/or external. Processing circuitry 500 may provide the data to be uplinked to external device 21 and the control signals for the telemetry circuit within communication circuitry 508, e.g., via an address/data bus. In some examples, communication circuitry 508 may provide received data to processing circuitry 500 via a multiplexer.

In some examples, ICM 300 may signal external device 21 to further communicate with and pass the alert through a network such as the Medtronic CareLink® Network developed by Medtronic plc., of Dublin, Ireland, or some other network linking patient 114 to a clinician. ICM 300 may spontaneously transmit information to the network or in response to an interrogation request from a user.

Power source 510 may be any type of device that is configured to hold a charge to operate the circuitry of ICM 300. Power source 510 may be provided as a rechargeable or non-rechargeable battery. In other example, power source 510 may incorporate an energy scavenging system that stores electrical energy from movement of ICM 200 within patient 114.

The various circuitry of IPD 16 may include any one or more processors, controllers, DSPs, ASICs, FPGAs, or equivalent discrete or integrated circuitry, including analog circuitry, digital circuitry, or logic circuitry.

PCD 110 may deliver ATP therapy and sense evoked response(s) at one or more locations and ICM 300 may sense evoked response(s) at one or more different locations via sensing circuitry 506. The evoked response(s) may be detected as described above with reference to FIG. 5.

According to the techniques of this disclosure, processing circuitry 500 may determine the occurrence of the cardiac event in patient 14. Processing circuitry 500 may determine, based at least in part on at least one accelerometer signal, at least one of a fall of patient 14, a slumping posture of patient 14, or a change from an upright posture to a non-upright posture of patient 14. Processing circuitry 500 may determine that the cardiac event and the at least one of the fall of patient 14, the slumping posture of patient 14, or the change from the upright posture to the non-upright posture of patient 14 are associated in time. In response to determining that the cardiac event and the at least one of the fall of patient 14, the slumping posture of patient 14, or the change from the upright posture to the non-upright posture of patient 14 are associated in time, processing circuitry 500 may modulate a response to the cardiac event.

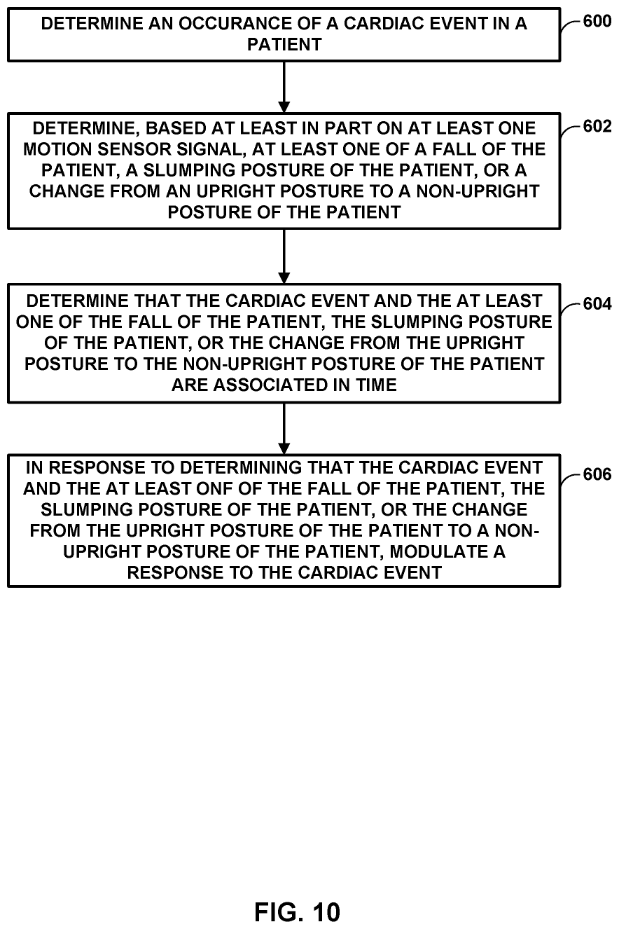

FIG. 10 is a flowchart illustrating example posture or fall based techniques for modulating a response to a detected cardiac event according to this disclosure. The discussion of FIG. 10 is in the context of system 8 for ease of explanation, however these techniques may be performed by any of the device described herein which are capable of performing such techniques.

System 8 may determine an occurrence of a cardiac event in patient 14 (600). For example, processing circuitry of system 8 may determine an occurrence of an arrythmia, pause in heartbeat, myocardial infarction, or other cardiac event in patient 14 based on sensor signals, such as ECG signals sensed by sensors of system 8.

System 8 may determine, based at least in part on at least one motion sensor signal, at least one of a fall of patient 14, a slumping posture of patient 14, or a change from an upright posture to a non-upright posture of patient 14 (602). For example, processing circuitry of system 8 may determine that one or more motion sensor (e.g., accelerometer) signals are indicative of a fall of patient 14, a slumping posture of patient 14, or a change from an upright posture to a non-upright posture of patient 14.

System 8 may determine that the cardiac event and the at least one of the fall of patient 14, the slumping posture of patient 14, or the change from the upright posture to the non-upright posture of patient 14 are associated in time (604). For example, processing circuitry of system 8 may compare a timing of the cardiac event to the timing of the fall of patient 14, the slumping posture of patient 14, or the change from the upright posture to the non-upright posture of patient 14 and determine that the cardiac event and the fall of patient 14, the slumping posture of patient 14, or the change from the upright posture to the non-upright posture of patient 14 when the fall of patient 14, the slumping posture of patient 14, or the change from the upright posture to the non-upright posture of patient 14 occurs during the cardiac event, during a first predetermined time period before the cardiac event, or during a second predetermined time period after the cardiac event.

In response to determining that the cardiac event and the at least one of the fall of patient 14, the slumping posture of patient 14, or the change from the upright posture to the non-upright posture of patient 14 are associated in time, system 8 may modulate a response to the cardiac event (606). For example, processing circuitry of system 8 may affect the treatment of patient 14 or the delivery or content of an indication relating to the cardiac event in response to the determination that the cardiac event and the at least one of the fall of patient 14, the slumping posture of patient 14, or the change from the upright posture to the non-upright posture of patient 14 are associated in time. For example, processing circuitry of system 8 may determine at least one of determine that the at least one of the fall of patient 14, the slumping posture of patient 14, or the change from the upright posture to the non-upright posture of patient 14 occurred during the cardiac event, within a first predetermined time period before the cardiac event, or within a second predetermined time period after the cardiac event.

In some examples, the cardiac event comprises at least one of a tachyarrhythmia, a pause in heartbeat, a stroke, or myocardial infarction. In some examples, modulating the response to the cardiac event comprises delivering treatment to patient 14. In some examples, the cardiac event comprises ventricular tachyarrhythmia and modulating the response to the cardiac event comprises overriding treatment rules and, delivering treatment to the patient includes, based on overriding the treatment rules, delivering a shock to patient 14 or delivering anti-tachyarrhythmia pacing therapy to patient 14. For example, processing circuitry of system 8 may override the treatment rules to deliver treatment sooner or later (e.g., at a different time) than system 8 would have delivered treatment if the treatment rules were not overridden.

In some examples, the cardiac event comprises ventricular tachyarrhythmia, and in response to the at least one of the fall of patient 14, the slumping posture of patient 14, or the change from an upright posture to a non-upright posture of patient 14, system 8 relaxes ventricular tachyarrhythmia determination criteria and determines that the cardiac event meets or surpasses the relaxed ventricular tachyarrhythmia determination criteria, wherein delivering treatment to patient 14 comprises delivering a shock to patient 14 based on determining that the cardiac event meets or surpasses the relaxed ventricular tachyarrhythmia determination criteria.

In some examples, prior to delivering the shock to patient 14, system 8 may determine that patient 14 has stopped moving for a predetermined period of time or that patient 14 is in the lying posture for the predetermined period of time; and in response to determining that patient 14 has stopped moving for a predetermined period of time or that patient 14 is in the lying posture for the predetermined period of time, delivering the shock to patient 14.

In some examples, the cardiac episode comprises an atrial tachyarrhythmia, and modulating the response to the cardiac episode comprises administering an anti-tachyarrhythmia pacing therapy sooner than otherwise (e.g., sooner than the medical device system would have administered the anti-tachyarrhythmia pacing therapy if the response was not modulated). In some examples, the cardiac event comprises an atrial tachyarrhythmia, and system 8 relaxes atrial tachyarrhythmia onset detection criteria to create relaxed atrial tachyarrhythmia onset detection criteria in response to the at least one of a fall of the patient, the slumping posture of the patient, or the change from an upright posture to a non-upright posture of the patient and determines that the cardiac event meets or exceeds the relaxed atrial tachyarrhythmia onset detection criteria.

In some examples, the modulated response to the cardiac event comprises generation of an indication for output, the indication comprising information indicative of the cardiac event and information indicative of at least one of the fall of the patient, the slumping posture of a patient, or the change from the upright posture to the non-upright posture of the patient. In some examples, system 8 may transmit the indication to one or more external devices. In some examples, transmitting the indication to the one or more external devices comprises: transmitting the indication to a first external device for further transmission to a second external device. In some examples, the one or more external devices comprise at least one of a patient device, a caregiver device, a call center device, a nursing home device, a remote monitoring system, or an emergency medical services device.

In some examples, prior to transmitting the indication to the one or more external devices, system 8 may determine a living situation of patient 14 and in response to the living situation of patient 14, determine to which of the one or more external devices to transmit the indication. For example, system 8 may transmit the indication based on the living situation of patient 14. In some examples, prior to transmitting the indication, system 8 may determine a user entered indication preference and transmit the indication in accordance with the user entered indication preference. In some examples, the user may be patient 14, a caregiver, or a clinician.

In some examples, the cardiac event comprises a pause in the heartbeat and the indication further comprises at least one of a) an indication of a first posture of interest prior to the cardiac event and an indication of a second posture of interest after the cardiac event, or b) an indication of whether a fall occurred during the cardiac event. In some examples, the first posture of interest is upright and the second posture of interest is non-upright or slumping.

In some examples, the cardiac event comprises a tachyarrhythmia, and wherein the indication for output comprises information indicative of how far (e.g., in terms of time) into the tachyarrhythmia the at least one of the fall of the patient, the slumping posture of a patient, or the change from the upright posture to the non-upright posture of the patient occurred.

In some examples, the cardiac event comprises a myocardial infarction. In such examples, system 8 may monitor an ST segment of an ECG. System 8 may determine that the ST segment more than a predetermined threshold higher than an average of earlier ST segments. In some examples, modulating the response to the cardiac event comprises sending the indication to an emergency medical services device. In some examples, in response to determining that the ST segment is more than the predetermined threshold higher than the average of earlier ST segments, system 8 may enable at least one of fall detection or posture detection.

In some examples, system 8 may send a communication to a cellular phone (which may be an example of external device 21) of patient 14. In some examples, in response to not receiving a response from patient 14 via the cellular phone, sending the indication to at least one of the one or more external devices (e.g., to the emergency medical services device).

In some examples, the motion sensor comprises an accelerometer. In some examples, the motion sensor is positioned in a pacemaker. In some examples, the pacemaker is a wholly intracardiac pacemaker.

Any suitable modifications may be made to the techniques described herein and any suitable device, processing circuitry, therapy delivery circuitry, and/or electrodes may be used for performing the steps of the methods described herein. The steps of the methods may be performed by any suitable number of devices. For example, a processing circuitry of one device may perform some of the steps while a therapy delivery circuitry and/or sensing circuitry of another device may perform other steps of the method, while communication circuitry may allow for communication needed for the processing circuitry to receive information from other devices. This coordination may be performed in any suitable manner according to particular needs.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, FRAM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to ICD 9, IPD 16, PCD 110, ICM 300, and external device 21, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. For example, any of the techniques or processes described herein may be performed within one device or at least partially distributed amongst two or more devices, such as between ICD 9, IPD 16, PCD 110, ICM 300, and/or external device 21. In addition, any of the described units, circuitry or components may be implemented together or separately as discrete but interoperable logic devices. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media.

As used herein, the term “circuitry” refers to an application specific integrated circuit (ASIC), an electronic circuit, a processor (shared, dedicated, or group) and memory that execute one or more software or firmware programs, a combinational logic circuit, or other suitable components that provide the described functionality.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

This disclosure includes the following non-limiting examples.

Example 1. A method comprising: determining, by a medical device system, an occurrence of a cardiac event in a patient; determining, by the medical device system and based at least in part on at least one motion sensor signal, at least one of a fall of the patient, a slumping posture of the patient, or a change from an upright posture to a non-upright posture of the patient; determining, by the medical device system, that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulating, by the medical device system, a response to the cardiac event.

Example 2. The method of example 1, wherein the cardiac event comprises at least one of a tachyarrhythmia, a pause in heartbeat, a stroke, or myocardial infarction.

Example 3. The method of example 1 or example 2, wherein modulating the response to the cardiac event comprises delivering treatment to the patient.

Example 4. The method of example 3, wherein the cardiac event comprises ventricular tachyarrhythmia, and modulating the response to the cardiac event comprises overriding treatment rules, wherein delivering treatment to the patient comprises, based on overriding the treatment rules, delivering a shock to the patient or delivering anti-tachyarrhythmia pacing therapy to the patient.

Example 5. The method of example 3, wherein the cardiac event comprises ventricular tachyarrhythmia, and wherein the method further comprises: in response to the at least one of the fall of the patient, the slumping posture of the patient, or the change from an upright posture to a non-upright posture of the patient, relaxing ventricular tachyarrhythmia determination criteria; and determining that the cardiac event meets or surpasses the relaxed ventricular tachyarrhythmia determination criteria, wherein delivering treatment to the patient comprises delivering a shock to the patient based on determining that the cardiac event meets or surpasses the relaxed ventricular tachyarrhythmia determination criteria.

Example 6. The method of example 4 or 5, further comprising: prior to delivering the shock to the patient, determining that the patient has stopped moving for a predetermined period of time or that the patient is in a lying posture for the predetermined period of time; and in response to determining that the patient has stopped moving for the predetermined period of time or that the patient is in the lying posture for the predetermined period of time, delivering the shock to the patient.

Example 7. The method of example 3, wherein the cardiac event comprises an atrial tachyarrhythmia, and modulating the response to the cardiac event comprises: administering an anti-tachyarrhythmia pacing therapy sooner than the medical device system would have administered the anti-tachyarrhythmia pacing therapy if the response was not modulated.

Example 8. The method of example 3, wherein the cardiac event comprises an atrial tachyarrhythmia, and wherein the method further comprises: relaxing atrial tachyarrhythmia onset detection criteria to create relaxed atrial tachyarrhythmia onset detection criteria in response to the at least one of a fall of the patient, the slumping posture of the patient, or the change from an upright posture to a non-upright posture of the patient; and determining that the cardiac event meets or exceeds the relaxed atrial tachyarrhythmia onset detection criteria.

Example 9. The method of example 1 or example 2, wherein the modulated response to the cardiac event comprises generation of an indication for output, the indication comprising information indicative of the cardiac event and information indicative of at least one of the fall of the patient, the slumping posture of a patient, or the change from the upright posture to the non-upright posture of the patient.

Example 10. The method of example 9, further comprising transmitting the indication to one or more external devices.

Example 11. The method of example 10, wherein transmitting the indication to the one or more external devices comprises transmitting the indication to a first external device for further transmission to a second external device.

Example 12. The method of example 10 or 11, wherein the one or more external devices comprise at least one of a patient device, a caregiver device, a call center device, a nursing home device, a remote monitoring system, or an emergency medical services device.

Example 13. The method of any of examples 10-12, further comprising: prior to transmitting the indication to the one or more external devices, determining, by the medical device system, a living situation of the patient; and in response to the living situation of the patient, determining to which of the one or more external devices to transmit the indication.

Example 14. The method of any of examples 10-13, further comprising: prior to transmitting the indication, determining a user entered indication preference; and transmitting the indication in accordance with the user entered indication preference.

Example 15. The method of example 14, wherein the user comprises the patient, a caregiver, or a clinician.

Example 16. The method of any of examples 9-15, wherein the cardiac event comprises a pause in the heartbeat and the indication further comprises at least one of a) an indication of a first posture of interest prior to the cardiac event and an indication of a second posture of interest after the cardiac event, or b) an indication of whether a fall occurred during the cardiac event.

Example 17. The method of any of examples 9-15, wherein the cardiac event comprises a tachyarrhythmia, and wherein the indication for output comprises information indicative of how far into the tachyarrhythmia the at least one of the fall of the patient, the slumping posture of a patient, or the change from the upright posture to the non-upright posture of the patient occurred.

Example 18. The method of any of examples 9-15, wherein the cardiac event comprises a myocardial infarction, further comprising: monitoring an ST segment of an electrocardiogram; determining that the ST segment more than a predetermined threshold higher than an average of earlier ST segments, wherein modulating the response to the cardiac event comprises sending the indication to an emergency medical services device.

Example 19. The method of example 18, further comprising in response to determining that the ST segment is more than the predetermined threshold higher than the average of earlier ST segments, enabling at least one of fall detection or posture detection.

Example 20. The method of example 18 or example 19, further comprising: sending a communication to a cellular phone of the patient; and in response to not receiving a response from the patient via the cellular phone, sending the indication to at least one of the one or more external devices.

Example 21. The method of any of examples 1-20, wherein the motion sensor comprises an accelerometer.

Example 22. The method of any of examples 1-21, wherein the motion sensor is positioned in a pacemaker.

Example 23. The method of example 22, wherein the pacemaker comprises a wholly intracardiac pacemaker.

Example 24. A medical device system comprising: memory configured to store information relating to an occurrence of a cardiac event in a patient; and processing circuitry configured to: determine the occurrence of the cardiac event in the patient; determine, based at least in part on at least one motion sensor signal, at least one of a fall of the patient, a slumping posture of the patient, or a change from an upright posture to a non-upright posture of the patient; determine that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulate a response to the cardiac event.

Example 25. The medical device system of example 24, wherein the cardiac event comprises at least one of a tachyarrhythmia, a pause in heartbeat, or myocardial infarction.

Example 26. The medical device system of example 24 or example 25, wherein the modulated response to the cardiac event comprises delivering treatment to the patient.

Example 27. The medical device system of example 26, wherein the cardiac event comprises ventricular tachyarrhythmia and as part of modulating the response to the cardiac event, the processing circuitry is configured to override treatment rules, wherein delivering treatment to the patient comprises, based on overriding the treatment rules, delivering a shock to the patient or delivering anti-tachyarrhythmia pacing therapy to the patient.

Example 28. The medical device of example 26, wherein the cardiac event comprises ventricular tachyarrhythmia, and wherein the processing circuitry is further configured to: in response to the at least one of a fall of the patient, the slumping posture of the patient, or the change from an upright posture to a non-upright posture of the patient, relax ventricular tachyarrhythmia determination criteria; and determine that the cardiac event meets or surpasses the relaxed ventricular tachyarrhythmia determination criteria, wherein delivering treatment to the patient comprises delivering a shock to the patient based on determining that the cardiac event meets or surpasses the relaxed ventricular tachyarrhythmia determination criteria.

Example 29. The medical device system of example 27 or 28, wherein the processing circuitry is further configured to: prior to delivering the shock to the patient, determine that the patient has stopped moving for a predetermined period of time or that the patient is in a lying posture for the predetermined period of time; and in response to determining that the patient has stopped moving for the predetermined period of time or that the patient is in the lying posture for the predetermined period of time, deliver the shock to the patient.

Example 30. The medical device system of any of example 26, wherein the cardiac event comprises an atrial tachyarrhythmia, and as part of modulating the response to the cardiac event, the processing circuitry is further configured to administer an anti-tachyarrhythmia pacing therapy sooner than the medical device system would have administered the anti-tachyarrhythmia pacing therapy if the response was not modulated.

Example 31. The method of example 26, wherein the cardiac event comprises an atrial tachyarrhythmia, and wherein the processing circuitry is further configured to: relax atrial tachyarrhythmia onset detection criteria to create relaxed atrial tachyarrhythmia onset detection criteria in response to the at least one of a fall of the patient, the slumping posture of the patient, or the change from an upright posture to a non-upright posture of the patient; and determine that the cardiac event meets or exceeds the relaxed atrial tachyarrhythmia onset detection criteria.

Example 32. The medical device system of example 24 or example 25, wherein the modulated response to the cardiac event comprises generation of an indication for output, the indication comprising information indicative of the cardiac event and information indicative of at least one of the fall of the patient, the slumping posture of a patient, or the change from the upright posture to the non-upright posture of the patient.

Example 33. The medical device system of example 32, wherein the processing circuitry is further configured to transmit the indication to one or more external devices.

Example 34. The medical device system of example 33, wherein as part of transmitting the indication to the one or more external devices, the processing circuitry is configured to transmit the indication to a first external device for further transmission to a second external device.

Example 35. The medical device system of example 33 or 34, wherein the one or more external devices comprise at least one of a patient device, a caregiver device, a call center device, a nursing home device, a remote monitoring system, or an emergency medical services device.

Example 36. The medical device system of any of examples 33-35, wherein the processing circuitry is further configured to: prior to transmitting the indication to the one or more external devices, determine a living situation of the patient; and in response to the living situation of the patient, determine to which of the one or more external devices to transmit the indication.

Example 37. The medical device system of any of examples 33-36, wherein the processing circuitry is further configured to: prior to transmitting the indication, determine a user entered indication preference; and transmit the indication in accordance with the user entered indication preference.

Example 38. The medical device of example 37, wherein the user comprises the patient, a caregiver, or a clinician.

Example 39. The medical device system of any of examples 32-38, wherein the cardiac event comprises a pause in the heartbeat and the indication for output further comprises at least one of a) an indication of a first posture of interest prior to the cardiac event and an indication of a second posture of interest after the cardiac event, or b) an indication of whether a fall occurred during the cardiac event.

Example 40. The medical device system of any of examples 32-38, wherein the cardiac event comprises a tachyarrhythmia, and wherein the indication for output comprises information indicative of how far into the tachyarrhythmia the at least one of the fall of the patient, the slumping posture of a patient, or the change from the upright posture to the non-upright posture of the patient occurred.

Example 41. The medical device system of any of examples 32-38, wherein the cardiac event comprises a myocardial infarction, wherein the processing circuitry is further configured to: monitor an ST segment of an electrocardiogram; determine that the ST segment more than a predetermined threshold higher than an average of earlier ST segments; and send the indication to an emergency medical services device.

Example 42. The medical device system of example 41, wherein the processing circuitry is further configured to in response to determining that the ST segment is more than the predetermined threshold higher than the average of earlier ST segments, enable at least one of fall detection or posture detection.

Example 43. The medical device system of example 41 or example 42, wherein the processing circuitry is further configured to: send a communication to a cellular phone of the patient; and in response to not receiving a response from the patient via the cellular phone, send the indication to at least one of the one or more external devices.

Example 44. The medical device of any of examples 24-43, wherein the motion sensor comprises an accelerometer.

Example 45. The medical device of any of examples 24-44, wherein the medical device comprises a pacemaker.

Example 46. The medical device of example 45, wherein the pacemaker comprises a wholly intracardiac pacemaker.

Example 47. The medical device of any of examples 24-46, 44. The medical device of claim 24, wherein as part of determining that the cardiac event and the at least one of the fall of the patient, the slumping posture of the patient, or the change from the upright posture to the non-upright posture of the patient are associated in time, the processing circuitry is configured to at least one of determine that the at least one of the fall of the patient, the slumping posture of the patient, or the change from the upright posture to the non-upright posture of the patient occurred during the cardiac event, within a first predetermined time period before the cardiac event, or within a second predetermined time period after the cardiac event.

Example 48. A non-transitory computer-readable storage medium having stored thereon instructions that, when executed, cause processing circuitry to: determine an occurrence of a cardiac event in a patient; determine, based at least in part on at least one motion sensor signal, at least one of a fall of the patient, a slumping posture of the patient, or a change from an upright posture to a non-upright posture of the patient; determine that the cardiac event and the at least one of the fall of the patient, the slumping posture of the patient, or the change from the upright posture to the non-upright posture of the patient are associated in time; and in response to determining that the cardiac event and the at least one of the fall of the patient, the slumping posture of the patient, or the change from the upright posture to the non-upright posture of the patient are associated in time, modulate, by a medical device system, a response to the cardiac event.

Various examples have been described for delivering cardiac stimulation therapies as well as coordinating the operation of various devices within a patient. Any combination of the described operations or functions is contemplated. These and other examples are within the scope of the following claims.

What is claimed is:

1. A medical device system comprising:
memory configured to store information relating to an occurrence of a cardiac event in a patient; and
processing circuitry configured to:
determine the occurrence of the cardiac event in the patient;
determine, based at least in part on at least one motion sensor signal, at least one of a fall of the patient, a slumping posture of the patient, or a change from an upright posture to a non-upright posture of the patient;
determine that the cardiac event and the at least one of the fall of the patient, the slumping posture of the patient, or the change from the upright posture to the non-upright posture of the patient are associated in time; and
in response to determining that the cardiac event and the at least one of the fall of the patient, the slumping posture of the patient, or the change from the upright posture to the non-upright posture of the patient are associated in time, modulate a response to the cardiac event.

2. The medical device system of claim 1, wherein the cardiac event comprises at least one of a tachyarrhythmia, a pause in heartbeat, myocardial infarction, or a stroke.

3. The medical device system of claim 1, wherein the modulated response to the cardiac event comprises delivering treatment to the patient.

4. The medical device system of claim 3, wherein the cardiac event comprises ventricular tachyarrhythmia and as part of modulating the response to the cardiac event, the processing circuitry is configured to override treatment rules, wherein delivering treatment to the patient comprises, based on overriding the treatment rules, delivering a shock to the patient or delivering anti-tachyarrhythmia pacing therapy to the patient.

5. The medical device of claim 3, wherein the cardiac event comprises ventricular tachyarrhythmia, and wherein the processing circuitry is further configured to:
in response to the at least one of a fall of the patient, the slumping posture of the patient, or the change from an upright posture to a non-upright posture of the patient, relax ventricular tachyarrhythmia determination criteria; and
determine that the cardiac event meets or surpasses the relaxed ventricular tachyarrhythmia determination criteria,
wherein delivering treatment to the patient comprises delivering a shock to the patient based on determining that the cardiac event meets or surpasses the relaxed ventricular tachyarrhythmia determination criteria.

6. The medical device system of claim 5, wherein the processing circuitry is further configured to:
prior to delivering the shock to the patient, determine that the patient has stopped moving for a predetermined period of time or that the patient is in a lying posture for the predetermined period of time; and
in response to determining that the patient has stopped moving for the predetermined period of time or that the patient is in the lying posture for the predetermined period of time, deliver the shock to the patient.

7. The medical device system of claim 3, wherein the cardiac event comprises an atrial tachyarrhythmia, and as part of modulating the response to the cardiac event, the processing circuitry is further configured to administer an anti-tachyarrhythmia pacing therapy sooner than the medical device system would have administered the anti-tachyarrhythmia pacing therapy if the response was not modulated.

8. The medical device system of claim 3, wherein the cardiac event comprises an atrial tachyarrhythmia, and wherein the processing circuitry is further configured to:
relax atrial tachyarrhythmia onset detection criteria to create relaxed atrial tachyarrhythmia onset detection criteria in response to the at least one of a fall of the patient, the slumping posture of the patient, or the change from an upright posture to a non-upright posture of the patient; and
determine that the cardiac event meets or exceeds the relaxed atrial tachyarrhythmia onset detection criteria.

9. The medical device system of claim 1, wherein the modulated response to the cardiac event comprises generation of an indication for output, the indication comprising information indicative of the cardiac event and information indicative of at least one of the fall of the patient, the slumping posture of a patient, or the change from the upright posture to the non-upright posture of the patient.

10. The medical device system of claim 9, wherein the processing circuitry is further configured to transmit the indication to one or more external devices.

11. The medical device system of claim 10, wherein the one or more external devices comprise at least one of a patient device, a caregiver device, a call center device, a nursing home device, a remote monitoring system, or an emergency medical services device.

12. The medical device system of claim 10, wherein the processing circuitry is further configured to:
prior to transmitting the indication to the one or more external devices, determine a living situation of the patient or a user entered indication preference; and
transmit the indication based on the living situation of the patient or in accordance with the user entered indication preference.

13. The medical device system of claim 9, wherein the cardiac event comprises a pause in the heartbeat and the indication for output further comprises at least one of a) an indication of a first posture of interest prior to the cardiac event and an indication of a second posture of interest after the cardiac event, or b) an indication of whether a fall occurred during the cardiac event.

14. The medical device system of claim 9, wherein the cardiac event comprises a tachyarrhythmia, and wherein the indication for output comprises information indicative of how far into the tachyarrhythmia the at least one of the fall of the patient, the slumping posture of a patient, or the change from the upright posture to the non-upright posture of the patient occurred.

15. The medical device system of claim 9, wherein the cardiac event comprises a myocardial infarction, wherein the processing circuitry is further configured to:
monitor an ST segment of an electrocardiogram;
determine that the ST segment more than a predetermined threshold higher than an average of earlier ST segments; and
send the indication to an emergency medical services device.

16. The medical device system of claim 15, wherein the processing circuitry is further configured to in response to determining that the ST segment is more than the predetermined threshold higher than the average of earlier ST segments, enable at least one of fall detection or posture detection.

17. The medical device system of claim 15, wherein the processing circuitry is further configured to:
send a communication to a cellular phone of the patient; and
in response to not receiving a response from the patient via the cellular phone, send the indication to at least one of the one or more external devices.

18. The medical device of claim 1, wherein as part of determining that the cardiac event and the at least one of the fall of the patient, the slumping posture of the patient, or the change from the upright posture to the non-upright posture of the patient are associated in time, the processing circuitry is configured to at least one of determine that the at least one of the fall of the patient, the slumping posture of the patient, or the change from the upright posture to the non-upright posture of the patient occurred during the cardiac event, within a first predetermined time period before the cardiac event, or within a second predetermined time period after the cardiac event.

19. A method comprising:
determining, by a medical device system, an occurrence of a cardiac event in a patient;
determining, by the medical device system and based at least in part on at least one motion sensor signal, at least one of a fall of the patient, a slumping posture of the patient, or a change from an upright posture to a non-upright posture of the patient;
determining, by the medical device system, that the cardiac event and the at least one of the fall of the patient, the slumping posture of the patient, or the change from the upright posture to the non-upright posture of the patient are associated in time; and
in response to determining that the cardiac event and the at least one of the fall of the patient, the slumping posture of the patient, or the change from the upright posture to the non-upright posture of the patient are associated in time, modulating, by the medical device system, a response to the cardiac event.

20. A non-transitory computer-readable storage medium having stored thereon instructions that, when executed, cause processing circuitry to:
determine an occurrence of a cardiac event in a patient;
determine, based at least in part on at least one motion sensor signal, at least one of a fall of the patient, a slumping posture of the patient, or a change from an upright posture to a non-upright posture of the patient;
determine that the cardiac event and the at least one of the fall of the patient, the slumping posture of the patient, or the change from the upright posture to the non-upright posture of the patient are associated in time; and
in response to determining that the cardiac event and the at least one of the fall of the patient, the slumping posture of the patient, or the change from the upright posture to the non-upright posture of the patient are associated in time, modulate, by a medical device system, a response to the cardiac event.
